(19) United States
(12) Patent Application Publication
(10) Pub. No.: US 2023/0000100 A1
(43) Pub. Date: Jan. 5, 2023

(54) FOOD PRODUCTS FROM PLANT PROTEIN ISOLATES

(71) Applicant: Climax Foods Inc., Berkeley, CA (US)

(72) Inventors: Oliver Zahn, Berkeley, CA (US); Malgorzata Jakubasch, Berkeley, CA (US); Daniel Westcott, Berkeley, CA (US); Alifia Merchant, Berkeley, CA (US)

(21) Appl. No.: 17/857,871

(22) Filed: Jul. 5, 2022

Related U.S. Application Data
(60) Provisional application No. 63/203,002, filed on Jul. 2, 2021.
(60) Provisional application No. 63/260,351, filed on Aug. 17, 2021.
(60) Provisional application No. 63/330,484, filed on Apr. 13, 2022.

(51) Int. Cl. A23C 20/02; A23C 11/10

(52) U.S. Cl. CPC A23C 20/025; A23C 11/103

(57) ABSTRACT

In variants, a food product can be made from protein assemblies having a higher-order protein structure formed from a plurality of protein oligomers. In an illustrative example, plant-based dairy analogs can be made from an emulsion of lipids and protein micelles, each including a higher-order structure formed from a plurality of protein oligomers, wherein each protein oligomer itself is formed from a set of protein units or chains.

### CROSS-REFERENCE TO RELATED APPLICATIONS

This application claims the benefit of U.S. Provisional Application No. 63/203,002 filed 2 Jul. 2021, U.S. Provisional Application No. 63/260,351 filed 17 Aug. 2021, and of U.S. Provisional Application No. 63/330,484 filed 13 Apr. 2022, each of which is incorporated in its entirety by this reference.

### TECHNICAL FIELD

This invention relates generally to the food analog field, and more specifically to a new and useful plant-derived protein assemblies in the food analog field.

### BRIEF DESCRIPTION OF THE FIGURES

FIG. 1 is a schematic representation of a variant of the method.

FIG. 2 depicts an example of the method.

FIG. 3 depicts a schematic example of a cross-section of a protein assembly.

FIG. 4 depicts an example of protein assembly emulsions at 0 hours and 15 hours.

FIG. 5 depicts a first illustrative example of Turbiscan Stability Index (TSI) data for protein assembly emulsions over time.

FIG. 6 depicts a second illustrative example of Turbiscan Stability Index (TSI) data for protein assembly emulsions over time.

FIG. 7 depicts an illustrative example of backscattering data for emulsions with protein assemblies showing a layer of fat separating from the emulsion after a substantial period of time.

FIG. 8 depicts an example of forming protein assemblies from protein isolates.

FIG. 9A depicts an illustrative example of a rheology time sweep test data for protein assembly gels.

FIG. 9B depicts an illustrative example of a rheology amplitude sweep test data for protein assembly gels.

FIGS. 10A, 10B, 10C, and 10D depict illustrative examples of Turbiscan data showing transmission (T) and backscattering (BS) intensities for protein assembly emulsions over time.

FIG. 11 depicts an illustrative example of texture profile analysis data for protein assembly gels.

FIG. 12 depicts an illustrative example of differential scanning calorimeter data for protein assembly gels.

### DETAILED DESCRIPTION

The following description of the embodiments of the invention is not intended to limit the invention to these embodiments, but rather to enable any person skilled in the art to make and use this invention.

### 1. Overview

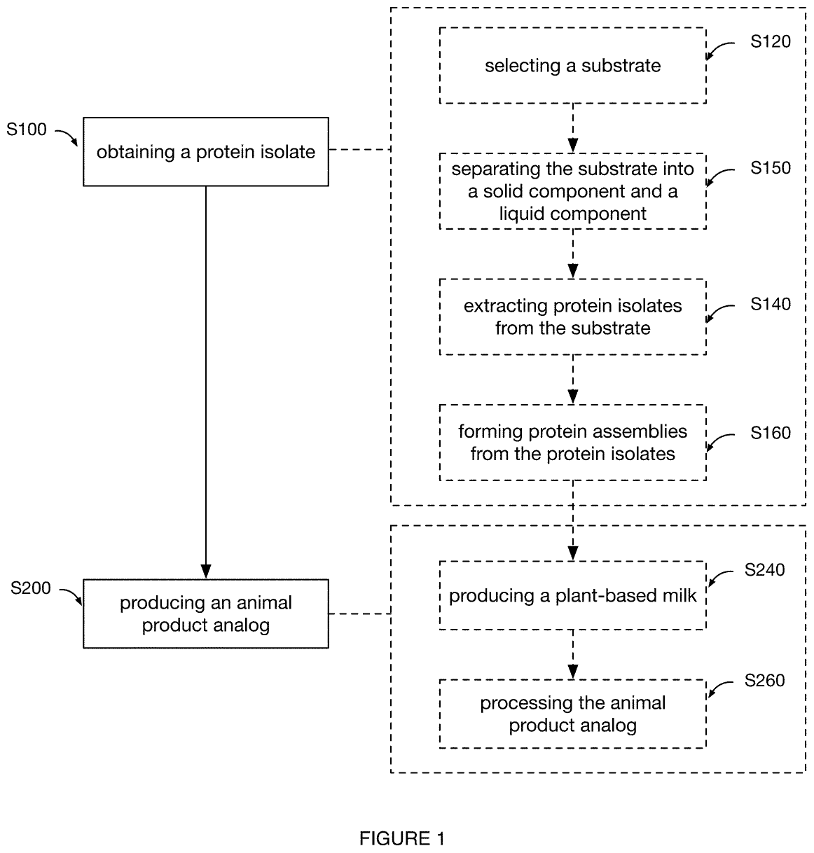

As shown in FIG. 1, the method can include: obtaining protein isolates S100, and producing an animal product analog S200.

In variants, the method can function to utilize protein assemblies (e.g., micelles of globulin, such as 11S globulin hexamers) to produce plant-based alternatives to animal products and/or provide other functionalities.

### 2. Technical Advantages

Variants of the technology can confer one or more advantages over conventional technologies.

First, conventional plant-derived milk analogs and downstream products thereof (e.g., butter, yogurt, cheese, etc.) are often achieved by using modifiers to augment properties of a plant-derived base until it resembles the animal product. Notably, this conventional approach does not satisfactorily match the functional behaviors of dairy milk or the dairy experience (e.g., emulsion stability, curdling behavior, flavor, whipping capacity, foaming capacity, texture properties of the resulting curds, etc.). Notable examples of this are plant-derived cheese products that often rely on gums and other texture modifiers, which may result in functional properties and protein contents that are distinct from those of dairy cheese. In variants, the inventors have discovered that forming protein assemblies, wherein the protein assemblies simulate functional properties of certain animal-derived proteins (e.g., increasing emulsion stability between a fatty component and an aqueous component), can improve the functional behaviors of a dairy analog product produced using the protein assemblies, which, in variants, can augment, reduce the need for, and/or eliminate the need for gums and/or other texture modifiers.

Second, in variants, the protein assemblies can scatter light as it passes through a protein assembly solution, wherein the protein assemblies can be white in color. Plant matter used as a base may not be a conventional milk color, such as, for instance, green in color. A spherical micelle structure of a protein assembly (e.g., with a diameter between 100-1000 nm) may diminish a green color of a milk analog solution containing the protein assembly by encapsulating the green matter and/or due to light scattering by the particles. Likewise, a plant-derived protein assembly solution can be “milk-white,” demonstrating light scattering as a result of assembly. Achieving the “milk-white” protein solution is a notable gain of function for plant-derived products since many plant extracts retain the color of the starting plant material.

Third, press cakes represent a potentially valuable waste byproduct of traditional oilseed extraction processes (e.g., cooking oil production; lipid extraction; etc.), particularly for plant-based alternatives to meat and/or dairy products. In examples, press cakes can be particularly useful since an additional lipid-removal step no longer needs to be performed prior to subsequent processing, and strong flavors within the substrate are oftentimes extracted with the lipid component (e.g., removed from the press cake). However, using press cakes in food production introduces several challenges, including matching nutritional content, flavor profile, and/or functional properties of a press cake food product to a target food product. The inventors have discovered that these challenges can be mitigated by reconstituting a press cake with secondary components (e.g., plant-based oil, creams, polysaccharides, etc.) from another source (e.g., foreign source, external source, etc.), and/or by extracting proteins from the press cake (e.g., for protein assembly formation). In an example, a fatty component can be selected and combined with the press cake such that the press cake substrate's flavor is masked, the nutritional profile more closely matches a target profile (e.g., including saturated fats), and/or to improve the functional property behavior of the food product.

However, further advantages can be provided by variants of the system and method disclosed herein.

### 3. Method

As shown in FIG. 1, the method can include: obtaining a protein isolate S100 and producing an animal product analog using the protein isolate S200. In variants, obtaining the protein isolate S100 can include: extracting protein isolates from a substrate S140, and forming protein assemblies from the protein isolates S160. Optionally, the method can include: selecting a substrate S120, separating the substrate into a solid component and a liquid component S150, producing a plant-based milk S240 (e.g., wherein the animal product analog is produced using the reconstituted product), processing the animal product analog S260, and/or any other suitable method.

The resultant animal product analog preferably has one or more functional properties similar to the target animal product. The resultant animal product analog is preferably entirely plant matter, but can additionally or alternatively be primarily plant matter (e.g., more than 50% plant matter; more than 99% plant matter; etc.), partially plant matter, and/or have any other suitable plant matter content. The resultant animal product analog can exclude and/or include less than a threshold amount of total and/or added: animal products (e.g., excludes animal proteins, such as caseins), gums (e.g., polysaccharide thickeners), allergenic ingredients, and/or any other suitable ingredient. Added ingredients and/or compounds can include: materials that were not present in and/or are foreign to the plant substrate or other ingredients, materials added in as a separate ingredient, and/or otherwise defined. The threshold amount can be between 0.1%-10% or any range or value therebetween (e.g., 10%, 5%, 3%, 2%, 1%, 0.1%, etc.), but can alternatively be greater than 10% or less than 0.1%. The animal product analog's functional properties that can be matched or substantially matched to the target animal product can include: nutritional profile (e.g., macronutrient profile, micronutrient profile, etc.), flavor, texture, phase change responses (e.g., melt, stretch, etc.), treatment responses (e.g., caramelization, etc.), and/or any other functional profile. For example, the animal product analog's functional properties can be matched to within a threshold difference from the target animal product, wherein the threshold difference can be between 1%-30% or any range or value therebetween (e.g., 1%, 5%, 10%, 30%, etc.), but can alternatively be greater than 30% or less than 1%. For example, a milk analog can have a protein percentage of about 3.5% by weight or volume, a soft cheese (e.g., cottage cheese, brie, etc.) analog can have a protein percentage of about 8%-21% by weight, a semi-hard cheese (e.g., cheddar) analog can have a protein percentage of about 18-28% by weight, and a hard cheese (e.g., gouda) can have a protein percentage of about 24-28% or higher by weight. Alternatively, the animal product analogs can have one or more functional properties that differ (e.g., drastically; more than 10%, 20%, 30%, 50%, and/or another percentile) from the target animal product.

Obtaining the protein isolate S100 functions to obtain a high-protein starting ingredient for animal product analog production. In variants, this can enable the resultant animal product analog to have functional properties (e.g., texture, phase change behavior, nutritional profile, etc.) that are similar to a target animal product, and/or confer other benefits.

One or more protein isolates can be obtained. When multiple protein isolates are obtained, the protein isolates can be of the same or different class (e.g., all globulins, etc.) or type (e.g., all 11S globulins, 11S globulins and 7S globulins, etc.), and/or can be from the same or different source/substrate (e.g., different protein isolates from the same plant and/or plant component; different protein isolates from different plants; etc.).

The protein isolates are preferably substantially pure (e.g., higher than 20%, 30%, 40%, 50%, 60%, 70%, 80%, 90%, 95%, 99%, etc.), but can alternatively be impure (e.g., have between 2%-10%, 5%-20%, 10%-30%, and/or any other suitable range of impurities. For example, the protein isolate can have a purity higher than 70%.

The proteins within the protein isolate can be of the same or different type. The proteins within the protein isolate can include: globulins (e.g., 2S globulins, 11S globulins, 7S globulins, conglutin, napin, sfa, edestin, amandin, concanvalin, vicilin, legumin, cruciferin, helianthinin, etc.), pseudoglobulins, globular proteins, prolamins, albumins, gluten, conjugated proteins (e.g., lipoprotein, mucoprotein, etc.), other storage proteins, animal proteins (e.g., casein, insect proteins, etc.), and/or any other suitable protein or combination thereof. In a specific example, the proteins include 80% edestin proteins, 19% vicilin proteins, and 1% other proteins. The proteins preferably are non-casein proteins, non-mammalian proteins, and/or non-animal proteins, but can alternatively include casein, mammalian, and/or animal proteins. For example, the proteins can include casein, mammalian, and/or animal proteins below a threshold amount, wherein the threshold amount can be between 0.1%-10% or any range or value therebetween (e.g., 10%, 5%, 3%, 2%, 1%, 0.1%, etc.), but can alternatively be greater than 10% or less than 0.1%. The sedimentation coefficient of the proteins can be between 6S-13S or any range or value therebetween (e.g., 7S-9S, 10S-12S, etc.), but can alternatively be less than 6S or greater than 13S. The solution of protein isolates may contain 0%-20% impurities or any range or value therebetween (e.g., 5%-15%, 10%, etc.), but can alternatively contain greater than 20% impurities. Impurities include other proteins, macronutrients, fats, molecules, particulates, and/or any other component. The protein isolates may optionally assemble together into protein aggregates (e.g., quinary structures, assemblies of quaternary structures, etc.). The protein isolates are preferably in an aqueous solution, but can additionally or alternatively be in an ionic solution (e.g., an aqueous solution with salts), nonpolar solution, a lipid solution (e.g., oil), and/or in any other solution. The protein isolates are preferably dissolved in the solution, but can alternatively be suspended within the solution or otherwise distributed within the solution.

In a first variant, the protein isolates form higher-order structures (e.g., protein-protein interactions forming agglomerates, and agglomerate-agglomerate interactions form quinary structures) wherein each higher order structure can be formed from one or more smaller protein subunits or chains. In some embodiments, a majority of the protein isolates in this variant do not form higher order structures (e.g., do not form micelles, homo-oligomers, and/or complexes) with other protein isolate units and/or higher-order structures (e.g., of the same or different type). For example, the protein isolate can include hexamers of 11s globulin units (e.g., 2 11s globulin units) which are not organized into higher order structures, or may only partially be organized into higher-order structures (e.g. quinary structures, micelles, agglomerates, complexes, or aggregates).

In a second variant, the protein isolates are aggregated into quinary structures (e.g., assemblies of quaternary structures; agglomerates, complexes, aggregates, etc.). In embodiments, each higher-order structure (e.g., quinary structure) includes two or more protein subunits (e.g., quaternary structures), wherein each protein subunit itself includes two or more proteins (e.g., protein subunits; tertiary structures). Examples of quinary structures that can be formed include: micelles, homoligomers, complexes, and/or any other quinary structure, aggregate, and/or agglomerate. For example, the protein isolates can include micelles formed by two or more oligomers (e.g., hexamers of ns globulin units). In another example, the protein isolates can include micelles of a combination of us globulin hexamers and 7s globulin trimers and/or hexamers. In another example, the protein isolates can include micelles formed by two or more us globulin oligomers (e.g., hexamers, trimers, etc.). The protein units within the higher-order structure (e.g., quinary structure) can be from a single plant protein source (e.g., exclusively sunflower seed protein isolates, exclusively hemp seed protein isolates, etc.), or multiple plant protein sources (e.g., a mixture of sunflower and rice protein isolates, etc.); include a single protein type (e.g., exclusively ns globulins) or a mixture of protein types (e.g., include both us globulins and one or more other proteins, such as 7s globulins, etc.); and/or be otherwise constructed. More than a threshold proportion of the protein isolate units in solution are preferably organized into higher-order (e.g., quinary) structures; alternatively, less than a threshold proportion of the protein isolate units in solution can be organized within a higher-order (e.g., quinary) structure. The threshold proportion of the protein isolate units can be between 20%-99% or any range or value therebetween (e.g., 20%, 30%, 40%, 50%, 60% 70%, 80%, 90%, 99%, etc.), but can alternatively be less than 20% or more than 99%. The higher-order (e.g., quinary) structures preferably only include protein units (e.g., exclude other components, such as sugars, starches, gums, fats, etc.) and optionally solvent (e.g., aqueous solution, water, etc.), but can additionally or alternatively include a threshold proportion of additional components (e.g., salts or ions, such as calcium ions; carbohydrates; vitamins; flavoring; etc.). The threshold proportion of additional components can be between 0%-40% or any range or value therebetween (e.g., less than 0.01%, 1%, 5%, 10%, 20%, 30%, 40%, etc.), but can alternatively be greater than 40%.

However, the protein isolates can have or be organized into any other suitable structure.

The protein isolate can be: obtained from a third party (e.g., in wet form, in dry form), extracted from a substrate, assembled, and/or otherwise obtained.

In a first variant, S100 includes obtaining the protein isolate from a third party. The protein isolate can be obtained in wet form, in dry form, and/or in any other suitable state.

In a second variant, the protein isolate can be extracted from a substrate S140, wherein the substrate can optionally be selected S120.

Selecting a substrate S120 can function to select a protein source and/or any other feedstock for the protein isolate. One or more substrates can be selected.

The substrate is preferably plant matter, but can alternatively be matter derived from insects, animals, fungi, and/or any other matter or combination thereof. Generally, the substrate may primarily include plant matter, such that the substrate may include over fifty percent plant matter. In some aspects, it may be desirable for the substrate to include over ninety-nine percent plant matter. It is feasible that the substrate includes exclusively plant matter. For instance, the substrate may include plant matter such as cocoa beans, truffles, olives, coconut flesh, grape pomace, pumpkin (e.g., pumpkin seed), cottonseed, canola, sunflower, hazelnut, pistachio, almond, walnut, crude walnut, cashew, brazil nuts, hazelnut, macadamia nuts, pecan, peanut, hemp, oat, rice, poppy, watermelon (e.g., watermelon seed), chestnut, chia, flax, quinoa, soybean, peas, cassava, citrus (e.g., citrus fiber), grape (e.g., grape pomace), split mung beans, aquafaba, lupini, fenugreek, kiwi, Sichuan pepper, mustard, sesame, sunflower seeds, algae, duckweeds (e.g., lenna); plants selected from the cucurbita, anacardium, cannabis, salvia, arachis, brassica, sesamun, legume, and/or other genuses; plants selected from the Anacardiaceae, Asteraceae, Leguminosae, Cucurbits, Rosaceae, Lamiaceae, and/or other family; and/or any other plant matter. The substrate may include major production oilseeds (e.g., soybean, rapeseed, sunflower, sesame, niger, castor, canola, cottonseed, etc.), minor production oilseeds (coconut, palm seed, pumpkin, etc.), and/or other crops or plant matter. The plant-derived proteins are preferably from one or more storage components of a plant (e.g., nuts, seeds, legumes, etc.), but can alternatively be derived from any other plant component (e.g., leaves, stems, roots, etc.). The substrate may include a single variety of plant matter, a mixture of various plant matter, include animal matter (e.g., insect matter, mammalian products, etc.), and/or include matter from any other source. In a first example, the substrate may include hemp and coconut flesh, such as, for instance, to include approximately 50% hemp by mass and 50% coconut flesh by mass. In a second example, the substrate may include a mixture that is 50% peas and 50% soybeans, or 30% almonds, 30% chia, and 40% lentils. However, the substrate can include any other matter.

The substrate (and/or components thereof) can optionally be selected based on the respective attributes of the substrate (and/or components thereof). The attributes may be selected for desirability in the substrate, desirability in a component of the substrate, and/or desirability in a product derived from the substrate. Examples of desirable attributes may include: cost of the substrate, nutritional content of the substrate (e.g., PDCAAS or DIAAS score), flavor of the substrate, functionalities of the substrate (e.g., functional properties, interaction with fat and/or other ingredients, etc.), appearance of the substrate (e.g., color), allergenicity of the substrate (e.g., a lower allergenicity), climate impact (e.g., carbon impact), allergenicity (e.g., wherein allergenic ingredients are excluded from the substrate mixture), and/or any other attribute. Functional properties can include: texture, mouthfeel, stretch, melt, gelation point, denaturation point, chemical properties, precipitation, emulsion stability, interactions with other molecules (e.g., dextrinization, caramelization, denaturation, coagulation, shortening, etc.), water binding capacity, stability in a given solvent, emulsion stability, foamability, whippability, flavor (or lack thereof), and/or any other properties.

Components of a substrate mixture maybe selected based on the individual, cumulative, and/or complementary characteristics of the components. For example, a first component may have a characteristic with different values than the characteristic of a second component. Characteristics can include: functional properties, nutrition (e.g., macronutrient profile, micronutrient profile, etc.), and/or other characteristics. The substrate mixture is preferably selected such that the resultant animal product analog's characteristics are substantially similar to (e.g., within a predetermined margin of error, such as 1%, 5%, 10%, 20%, 30%, etc.) that of the target animal product, but can alternatively be otherwise selected. In an example, characteristics may include a protein profile of the components. For instance, it may be desirable to create a substrate with a specified protein profile. The first component of a substrate mixture may be selected for having aspects of a protein profile that, when combined with the second component, wherein the second component may have a protein profile that complements the protein profile of the first component, the protein profile of the mixture may substantially match the specified protein profile. Put another way, the first component may include aspects of a desired protein profile, but may be missing certain desired proteins. The second component may be selected based on including the missing desired proteins, thus allowing the substrate mixture to have the full desired protein profile.

The substrate and/or components thereof can optionally be selected as those with the highest attribute values, based on an optimization (e.g., between nutrition, cost, extraction difficulty, etc.), based on a collective attribute profile (e.g., select complimentary substrate components that collectively produce a desired substrate nutritional profile, etc.), and/or otherwise selected. In a first example, a set of substrates and/or substrate components can be selected to collectively include all or a plurality of proteins, precursors to protein analogs, and/or amino acids within milk and/or another target protein profile. In a second example, a substrate is selected based on the globular protein content (e.g., pumpkin seeds may be selected due to high globular protein content). In an illustrative example, the substrates high in 11s globulins and/or 7s globulins can be selected for inclusion. In a third example, the addition or enrichment of lipid-binding proteins may decrease creaming. Enzymatically-treated proteins may be used that may act as hydrophobic anchors on the end, which may increase lipid affinity. Further combinations of ingredients may accomplish texture functionalities, such as milk analog stability, which may incorporate, for example, specific gelling and/or emulsifying proteins or fats that may have the effect of, for instance, creating saturated fat to create certain room temperature stability.

However, the substrate can be otherwise selected.

The substrate can be whole (e.g., with all macronutrients and/or components intact), be sourced with a component removed from the substrate (e.g., lipid-removed, dried, etc.), and/or be sourced in any other suitable format.

In a first variant, the substrate is lipid-removed, wherein a portion of oils, fats, and/or flavor compounds have been removed from the original form of the substrate (e.g., the natural form of the substrate, the whole substrate). The lipid can be removed from the whole substrate by a third party (e.g., wherein the substrate is obtained from the third party) and/or removed by any other entity. In this variant, the substrate can be: a press cake, a waste byproduct from an oilseed extraction process, and/or any other product. In this variant, the substrate can include less than: 80%, 70%, 60%, 50%, 40%, 30%, 20%, 10%, 5%, 1% 0.01%; between 1% and 80%, between 0% and 10%, between any of the values discussed above; and/or any other proportion lipid component by weight (e.g., dry weight, liquid weight, etc.) or volume. In an illustrative example, the substrate can include less than 30% lipid content by weight. In another illustrative example, the substrate can include less than 20% lipid content by weight. In another illustrative example, the substrate can include less than 15% lipid content by weight. In another illustrative example, the substrate can include between 6%-9% lipid content by weight. Additionally or alternatively, less than: 80%, 70%, 60%, 50%, 40%, 30%, 20%, 10%, 5%, 1% 0.01%; any other suitable range therein, and/or any other proportion of flavor compounds and/or aromatics can be removed from the original (whole) substrate. In variants, a lipid-removed substrate can be a desirable starting substrate, due to the relative proportions of macronutrients (e.g., proteins, fats, carbohydrates, etc.) in the lipid-removed material and/or the reduced cost of using the lipid-removed substrate, since an additional lipid-removal step can be skipped or drastically minimized. For example, the amount of protein in the lipid-removed substrate can be substantially higher (e.g., 3 times higher) than the amount of fat in the lipid-removed substrate, which can enable easier, purer, and/or higher-concentration protein extraction. One or more ingredients and/or compounds can be derived (e.g., extracted, processed, etc.) from the lipid-removed substrate. Examples of ingredients and/or compounds that can be derived from the lipid-removed substrate can include: protein extracts, carbohydrate extracts, sugar extracts, fiber extracts, flavor extracts, nutrient extracts, solid matter (e.g., powders, fibers, etc.), and/or any other suitable ingredient, extract, and/or other product.

In a second variant, the substrate is whole (e.g., does not have any macronutrients extracted from the substrate).

In a first embodiment, the substrate can be used in its whole form for subsequent processes. For example the whole substrate can be comminuted and the particulates added to the final composition.

In a second embodiment, proteins can be extracted from the whole substrate, wherein the proteins can be used in subsequent processes. In this embodiment, other extracts, such as fats, sugars, and/or other components can be left within the protein extract and/or removed from the protein extract (e.g., to form a protein isolate).

In a third embodiment, the method can optionally include separating the whole substrate into a solid component and a liquid component S150, which functions separate oils from the substrate (e.g., remove or extract lipids from the substrate). S150 can be performed prior to S140 and/or at any other time. Separating the substrate into a solid component and a liquid component can include subjecting the substrate to mechanical processing, chemical processing, and/or any other separation processing. For instance, the substrate may be pressed, such that mechanical force is applied to the substrate. Any method of pressing may be used, such as screw pressing and compaction. The separation processing functions to extract the oils from the substrate, such that the remaining solid components of the substrate may be substantially free of oils. The solid component of the substrate may have a proportion of oil (or any fat) content that is 0%-30% or any range or value therebetween (e.g., less than 15%, less than 10%, less than 5%, etc.) by weight and/or volume, but can alternatively have a proportion of oil content that is greater than 30%. The remaining solid components of the substrate maybe considered a “press cake.” The press cake maybe formed into a shape, or otherwise discretized into a press cake unit.

However, the substrate can be otherwise pre-processed.

Extracting protein isolates from the substrate S140 functions to produce a concentrated protein solution from the substrate and/or component thereof. S140 can be performed after S120, after S150, and/or at any other time.

Extracting protein isolates S140 can include: producing an aqueous mixture using the substrate, adjusting a salt concentration of the aqueous mixture, adjusting a pH level of the aqueous mixture, collecting protein isolates from the aqueous mixture, and/or any other suitable process.

Producing an aqueous mixture using the substrate can include comminuting the substrate (e.g., the solid component of the substrate) and diluting the substrate with an aqueous solution to form the aqueous mixture. The aqueous solution is preferably a salt water solution, but can alternatively be water (e.g., wherein salts are optionally added after dilution) and/or any other aqueous solution. The substrate can be concurrently comminuted while diluting with the aqueous solution (e.g., a press cake and water are blended together), comminuted prior to diluting in an aqueous solution, comminuted after diluting in an aqueous solution, and/or comminuted at any other time. Any specific manner of comminution may be used, including: pulverizing, blending, crushing, tumbling, crumbling, atomizing, shaving, grinding, milling, cryo-milling, chopping, and/or any other comminution method. In a specific example, comminuting a press cake substrate can create press cake particulates having a predetermined size or size distribution, which can optionally be used as a component within the reconstituted product (e.g., S240). Any suitable equipment for carrying out any of these processes can also be used.

Adjusting the salt concentration of the aqueous mixture can function to solubilize and/or extract proteins from the substrate, stabilize the proteins, cause the proteins to form desired tertiary, quaternary, quinary, and/or other structures, and/or confer other functionalities. Adjusting the salt concentration of the aqueous mixture can be performed prior to producing the aqueous mixture using the substrate (e.g., adjusting the salt concentration of the aqueous solution), performed after producing the aqueous mixture (e.g., adding salts to the mixture), and/or performed at any other time. Adjusting the salt concentration can include increasing or decreasing the concentration of salt in the aqueous mixture (and/or components therein) to achieve a target salt concentration. The target salt concentration can be between 0.5-25,000 millisiemens or any range or value therebetween (e.g., 1-10 millisiemens, 2-8 millisiemens, 100-10,000 millisiemens, etc.), but can alternatively be less than 0.5 millisiemens or greater than 25,000 millisiemens, 0.1%. 0.2%, 0.3%, 0.5%, and/or any other percentage of the aqueous solution and/or mixture by weight or volume; and/or have any other concentration. In an example, increasing the concentration of salt includes adding salts to the aqueous mixture (e.g., adding salts to the aqueous solution prior to diluting the substrate with the aqueous solution, adding salts to the aqueous mixture, etc.). Examples of salts include: NaCl, calcium citrate, CaCl₂), sodium citrate, MgCl₂, salts with cations (e.g., divalent cations, such as calcium), and/or any other salt. The ionic strength of the aqueous mixture may be selected based upon the desired yield or functional properties of the desired protein isolate, which can be substrate specific. For example, when producing a fava bean protein isolate, it may be desirable to use an aqueous solution with an ionic strength of approximately 0.5M NaCl during protein isolate extraction, while it may be desirable to use an ionic strength of approximately 0.9M NaCl when producing a pumpkin seed isolate. However, the salt concentration can be otherwise adjusted.

Adjusting the pH level of the aqueous mixture can function to solubilize or extract proteins from the substrate, stabilize the proteins, cause the proteins to form desired tertiary, quaternary, and/or other structures, and/or confer any other functionality. The pH adjustment can be performed prior to producing the aqueous mixture using the substrate (e.g., before adding salts to the adjusting the pH level of the aqueous solution), performed after producing the aqueous mixture, while producing the aqueous mixture, while adjusting the ionic strength of the aqueous mixture, and/or performed at any other time. Adjusting the pH level can include adding one or more acids and/or bases to bring the pH of the aqueous mixture (and/or components therein) to a target pH. Examples of acids and bases that can be used include: HCl, NaOH, citric acid, lactic acid, gluconic acid, and/or any food-safe (e.g., at relevant quantities) acid and/or base. The target pH is preferably basic (e.g., greater than 7, greater than 8, greater than 9, etc.), but can alternatively be acidic and/or neutral. For example, the target pH can be between 4-11.5 or any range or value therebetween (e.g., 7-10, 8-9, 9-10, 9.4-9.6, etc.), but can alternatively be less than 4 or greater than 11.5. For example, the pH level of the aqueous mixture may be adjusted to approximately 9.5 by adding to the aqueous mixture (or any component thereof) any number of bases that are known to increase the alkalinity of a substance, such as a sodium hydroxide solution. However, the pH level can be otherwise adjusted.

Collecting protein isolates from the aqueous mixture functions to obtain proteins having a predetermined structure and/or purity. Collecting the protein isolates can include: separating non-soluble mixture components (e.g., proteins, fats, cellular debris, etc.) from the aqueous mixture and collecting soluble protein isolates within the aqueous phase. However, the protein isolates can be otherwise collected from the aqueous mixture.

In a first example, separating non-soluble mixture components from the aqueous mixture includes centrifuging the aqueous mixture to separate the aqueous mixture into soluble and insoluble components, wherein each of the soluble and insoluble components may include the macromolecule components of the aqueous mixture (e.g., nucleic acids, carbohydrates, lipids, proteins, etc.). In a second example, separating non-soluble mixture components includes filtering the mixture (e.g., using a mesh colander). In other examples, separating non-soluble mixture components includes decanting, drying, allowing sedimentation to occur, chilling, and/or any other separation method.

The soluble components in the aqueous phase (e.g., the supernatant) may then be selected from the aqueous mixture, wherein the aqueous phase solution may be a liquid, such as water, with protein isolates (e.g., soluble protein isolates) and/or protein isolate precursors. Collecting the soluble protein isolates can include: collecting the aqueous phase, retaining the aqueous phase, and/or otherwise treating the aqueous phase. The protein concentration (by weight) of the aqueous phase can be between 1%-50% or any range or value therebetween (e.g., 3%-10%, 5%-40%, 10%-15%, 15%-25%, etc.). Alternatively, the protein concentration can be less than 1% or greater than 50%. In variants, the soluble protein isolates within the aqueous phase can be collected without an additional fat removal step (e.g., wherein the aqueous phase is not chilled to assemble the lipids in solution). In embodiments, this can be accomplished by extracting the protein isolate from a lipid-removed or lipid-reduced substrate. Alternatively, the aqueous phase can be repeatedly treated (e.g., chilled, filtered, reacted, etc.) to remove one or more components (e.g., proteins, fats, starches, etc.). In an illustrative example of the alternative, the aqueous phase can be repeatedly chilled for fat removal (e.g., as disclosed in CA2244398, incorporated herein in its entirety by this reference). However, non-target-protein components can be otherwise removed.

However, protein isolates can be otherwise extracted.

However, the protein isolates can be otherwise obtained.

Forming protein assemblies from the protein isolates S160 functions to arrange proteins into a desired and/or predetermined higher-order protein structure (e.g., quaternary structure, quinary structure, etc.). Examples of higher-order protein structures that can be formed include: quaternary structures (e.g., hexamers, trimers, octamers, etc.), quinary structures, other higher-order structures (e.g., senary, septenary, octonary, nonary, denary, etc.), oligomers, homoligomers, complexes, and/or any other higher-order protein structure. For example, S160 can form micelles of 11S globulin hexamers, wherein the 11S globulin hexamers can be extracted from the substrate in S140. As used herein, each successive higher-order protein structure can include assemblies, aggregates, agglomerates, and/or associations of multiple protein subunits of the preceding order, or can be otherwise defined. For example, a quinary structure can be or include an agglomeration, assembly, or association of several quaternary structures in a closely packed arrangement, wherein each quinary structure subunit can have its own primary, secondary, tertiary, and/or quaternary structure. The quinary subunits can be held together by hydrogen bonds, London dispersion forces, van der Waals forces, and/or any other suitable chemical bond. However, the quinary structure can be otherwise defined. However, higher-order protein structures can be otherwise defined.

The protein assemblies (e.g., micellar protein isolates (MPIs)) can be formed from a single protein isolate (e.g., an isolate of a single protein type), multiple protein isolates (e.g., isolates of multiple different protein types), and/or any other set of protein isolates. The assemblies can be agglomerates, aggregates, higher-order structures, and/or any other suitable association between protein units. When multiple protein isolates are used, the protein isolates can be from a single substrate (e.g., single plant source), multiple substrates (e.g., multiple plant sources, multiple forms of a single plant source, etc.), and/or any other combination of substrates. For example, a first protein isolate solution and a second protein isolate solution (e.g., produced from a different substrate through a process optimized for the different substrate) may be combined. The combined protein isolate solution may then be used to form the protein assemblies. For instance, one or more protein isolate solutions maybe combined prior to forming the protein assemblies to form protein assemblies with unique compositions. Alternatively, isolated protein assemblies formed from different protein isolate solutions maybe combined after or during protein assembly formation to create mixed populations of protein assemblies. A combination of protein isolate solutions may be selected based on complementary characteristics of the protein isolate solutions. For instance, it maybe desirable to form a protein assembly that includes a specified protein profile (e.g., size, denaturation points, surface hydrophobicity, lipid binding, etc.). This may be a protein profile that is present in a dairy milk and/or other dairy product, a target protein profile (e.g., a 50/50, 40/60, 30/70, 20/80, 10/90, 5/95, 95/5, 90/10, 80/20, 70/30, 60/40, and/or any other ratio between a first and second protein isolate), and/or any other protein profile. Alternatively, a specific protein profile may be found to have benefits in the assembly formation process distinct from that found in dairy. The protein isolate solution is preferably substantially pure protein, but can additionally or alternatively include impurities (e.g., fats, salts, starches, sugars, etc.), additives (e.g., fats, salts, starches, sugars, flavoring, etc.), and/or any other suitable component. In a first example, the combined protein isolate solution can include nS globulins from a first and second substrate (e.g., pumpkin seed and hemp seed) in an approximately 50/50 ratio. In a second example, the combined protein isolate solution can include globulins (e.g., 11S globulins) and sugars. In a third example, the combined protein isolate solution can include the protein isolates and calcium (e.g., in comparable proportion as the calcium concentration in casein). However, the combined protein isolate solution can include any other suitable set of compounds.

In variants, forming protein assemblies includes: diluting the protein isolates and optionally concentrating the diluted protein isolates (e.g., the resultant protein assemblies). However, the protein assemblies can be otherwise formed.

Diluting the protein isolate solution functions to assemble the protein isolates into higher-order protein structures. In an example, this can form micelles (e.g., assemblies; higher-order protein structures) of protein oligomers. In an illustrative example, rapid dilution can cause proteins to aggregate and/or assemble into micelles by rapidly changing the isoelectric environment surrounding each protein (e.g., wherein the micelle structure is maintained by inter-protein and/or inter-protein oligomer interactions, such as ionic bonds, van der Waals bonds, hydrogen bonds, etc.; and/or by differences between the interior and exterior isoelectric points). Alternatively, dilution can cause protein aggregation or assembly into a micellar structure via any other suitable mechanism.

Diluting the protein isolates preferably includes using an aqueous solution, such as water (e.g., deionized water), to dilute the protein isolates, but can alternatively use organic and/or inorganic solvents (e.g., alcohol) to dilute the protein isolates. The protein isolate is preferably diluted to a target concentration rapidly (e.g., immediately; all of the solvent is added to the protein isolate solution at once), but can alternatively be diluted incrementally or at any other rate. The solvent (e.g., water) used to dilute the aqueous phase can be between 2° C.-35° C. or any range or value therebetween (e.g., 3° C.-15° C., 20° C.-25° C., be room temperature, etc.), but can alternatively be less than 2° C. or greater than 35° C. In an example, the solvent may be added to the protein solution at a ratio of between 1:1-10:1 (liquid to protein isolate solution by volume or mass) or any range or value therebetween (e.g., 2:1-7:1, 3:1-5:1, etc.), but can alternatively be added at a ratio less than 1:1 or greater than 10:1. However, diluting the protein isolates can be otherwise performed.

Concentrating the diluted protein isolates can function to concentrate protein assemblies (e.g., formed from the protein isolates during dilution) and/or to instigate formation of the protein assemblies. The protein concentration in the resulting protein assemblies (e.g., in each micelle) can be between 10%-79% (by weight) or any range or value therebetween (e.g., 15%-50%, 25%-45%, etc.), but can alternatively be less than 10% or greater than 79%.

In a first variant, concentrating the diluted protein isolates includes allowing sedimentation to occur and collecting the sediment, wherein the sediment includes the protein assemblies. This can include resting the diluted protein isolate (allowing sedimentation to occur) for a predetermined time period. The time period can be between 10 minutes to 48 hours, be any range or value therebetween (e.g., 1 hour-24 hours, 4 hours-20 hours, etc.), but can alternatively be allowed to rest for less than 10 minutes or greater than 48 hours. While sedimentation is occurring, the diluted protein isolate may be maintained at a temperature of −80° C.-30° C. or any range or value therebetween (e.g., 0° C.-12° C., 4° C., room temperature, etc.), but can alternatively be maintained at a temperature less than −80° C. or greater than 30° C.

In a second variant, concentrating the diluted protein isolates includes centrifuging the diluted protein isolate solution. For example, the diluted protein isolate solution can be centrifuged for 1 minute-1 hour or any range or value therebetween (e.g., 10 minutes-30 minutes, 20 minutes, etc.), but can alternatively be centrifuged for less than 1 minute or greater than 1 hour. Centrifugation can occur at 300 rpm-15000 rpm or any range or value therebetween (e.g., 5000 rpm), but can alternatively be centrifuged at less than 300 rpm or greater than 15000 rpm. The pellet is collected after centrifugation, wherein the pellet includes the protein assemblies.

In a third variant, concentrating the diluted protein isolates includes allowing sedimentation to occur and subsequently further concentrating the protein isolates (e.g., via centrifugation, filtration, or dialysis). In this variant, the pellet is collected after centrifugation, wherein the pellet includes the protein assemblies.

The resulting protein assembly (e.g., after concentration) can be a solid, a liquid (e.g., a viscous liquid), a gel (e.g., pre- or post-heating), and/or any other mixture type. Optionally, the protein assemblies can be diluted (e.g., diluted with water, any aqueous solution, oil, etc.). Dilution can function to achieve a desired protein concentration, a desired protein assembly solution consistency, a desired salt concentration (e.g., wherein the protein assembly solution can be diluted with water and subsequently reconcentrated to reduce salt in the protein assembly solution), and/or for any other function for downstream use.

In an example, the protein assemblies can be formed using the method disclosed in CA 2,244,398, which is incorporated in its entirety by this reference. In another example, the protein assemblies are formed by diluting the protein isolate (e.g., at room temperature, with or without repeated fat removal steps).

However, the diluted protein isolates can be otherwise concentrated.

However, the protein assemblies can be formed by: emulsifying the protein isolate with oil and acidifying the emulsion (e.g., wherein micelles containing the protein and optionally oil are formed responsive to the acidification); adding calcium to the protein isolate solution, wherein protein-calcium interaction forms the micelles; adding an edible surfactant to the protein isolate solution, wherein emulsification and/or pressurized protein isolate injection through the surfactant forms the micelles; and/or otherwise formed.

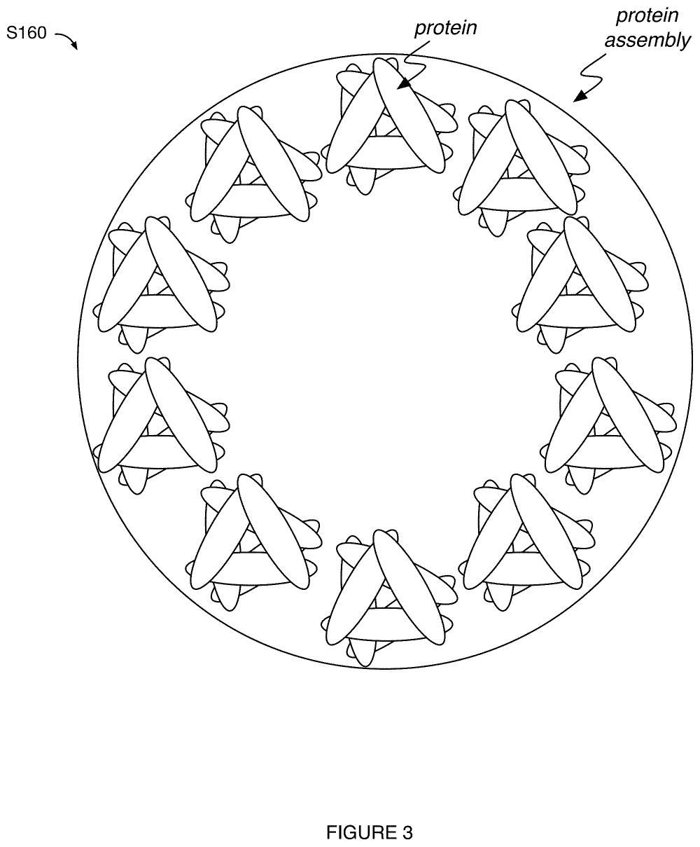

The protein assemblies are preferably micelles (e.g., reverse micelles and/or standard micelles), but can alternatively be any other protein assembly, aggregate, and/or agglomerate. The protein assemblies can include a set of protein oligomers (e.g., tertiary protein structures) arranged into a sphere (e.g., a micelle), but can alternatively have any other structure. The protein oligomers are preferably hexamers, but can alternatively be dimers, trimers, tetramers, pentamers, and/or any other oligomer. The diameter of the protein oligomers can be between 10 nm-200 nm or any range or value therebetween (e.g., 20 nm-100 nm, 40 nm, etc.), but can alternatively be less than 10 nm and/or greater than 200 nm. The diameter of the protein assembly (e.g., the quaternary structure, the micelle) can be between 50 nm-5000 nm or any range or value therebetween (e.g., 100 nm-500 nm, 100 nm-300 nm, 180 nm, 210 nm, etc.), but can alternatively be less than 50 nm or greater than 5000 nm. The micelles may form interconnecting structures that are known as “supermicelles.” Additionally, the micelles maybe more akin to casein micelles (e.g., calcium-rich protein aggregates with internal, water-filled, cavities) and/or other animal-based proteins. An example depicting a cross-section of a protein assembly is shown in FIG. 3.

The protein oligomers, the protein units forming the protein oligomers, and/or the protein assemblies preferably have both hydrophilic and hydrophobic regions, but alternatively can have mostly or entirely hydrophilic or hydrophobic regions (e.g., multiple hydrophilic regions with differing degrees of hydrophilicity, regions with similar hydrophilicity/hydrophobicity, etc.). In a first variant, the protein assemblies are micelles with a first face that is hydrophilic and a second face that is hydrophobic. For example, the micelles may have an external face, wherein the external face is in contact with the solution in which the micelle is suspended, and an internal face, wherein the internal face is substantially opposed to the external face. In a first example, the external face of the micelle may be hydrophilic and the internal face of the micelle may be hydrophobic. In a second example, the external face of the micelle maybe hydrophobic and the internal face of the micelle maybe hydrophilic, such that the micelle maybe a “reverse micelle.” In a second variant, the protein oligomers within the protein assemblies (e.g., higher-order structure; quinary structure; etc.) have polar (hydrophilic) regions between the protein oligomers (e.g., such that the interaction between two protein oligomers includes interactions between a negative charge region on one oligomer and a positive charge region on the other oligomer). In this variant, the regions of the oligomers on the external and/or interface face of the micelle may be more nonpolar (e.g., more hydrophobic) than the regions between oligomers, but can alternatively be less polar. In a third variant, the protein oligomers within the protein assemblies (e.g., higher-order structure; quinary structure; etc.) have nonpolar (hydrophobic) regions between the protein oligomers (e.g., such that the interaction between two protein oligomers includes interactions between nonpolar regions of the two oligomers). In this variant, the regions of the oligomers on the external and/or interface face of the micelle may be more polar (e.g., more hydrophilic) than the regions between oligomers, but can alternatively be less polar. In a fourth variant, the orientation of protein oligomers within the protein assemblies (e.g., higher-order structure; quinary structure; etc.) may change depending on the surrounding environment. In a first example, the protein assembly is a reverse micelle when substantially surrounded by oil, and a standard micelle when substantially surrounded by water. In a second example, the protein oligomers may be oriented with hydrophobic regions positioned between neighboring oligomers (within the protein assembly) when the protein assembly is immersed in water, and the protein oligomers may be oriented with hydrophobic regions positioned towards the internal and/or external face of the protein assembly when a fatty component is introduced. However, the hydrophilic and/or hydrophobic regions of the protein oligomers and/or protein assemblies maybe otherwise oriented.

Additional components and/or ingredients can optionally be added to the protein assembly solution (e.g., before, during, or after S240, etc.). The addition of components to the protein assembly solution (e.g., the fatty component, additional ingredients, etc.; described in S240) can optionally modify the nature, composition, and/or orientation of the protein assemblies (e.g., higher-order structure; quinary structure; etc.) and/or protein oligomers therein. For example, the addition of a fatty component may increase protein/lipid associations of certain hydrophobic protein domains, resulting in a more stable colloidal solution. Additionally, the addition of components can optionally change the size of the protein assemblies (e.g., a lipid/protein assembly) and/or the net surface charge of the protein assembly (e.g., by varying salt concentrations or pH). Such changes may include the size of the protein assembly spheres, the number of protein oligomers within a protein assembly, the temperature stability of the protein assemblies, the interactions within and between protein assemblies, nutritional availability, digestibility of the proteins within the protein assemblies, and/or any other protein assembly property.

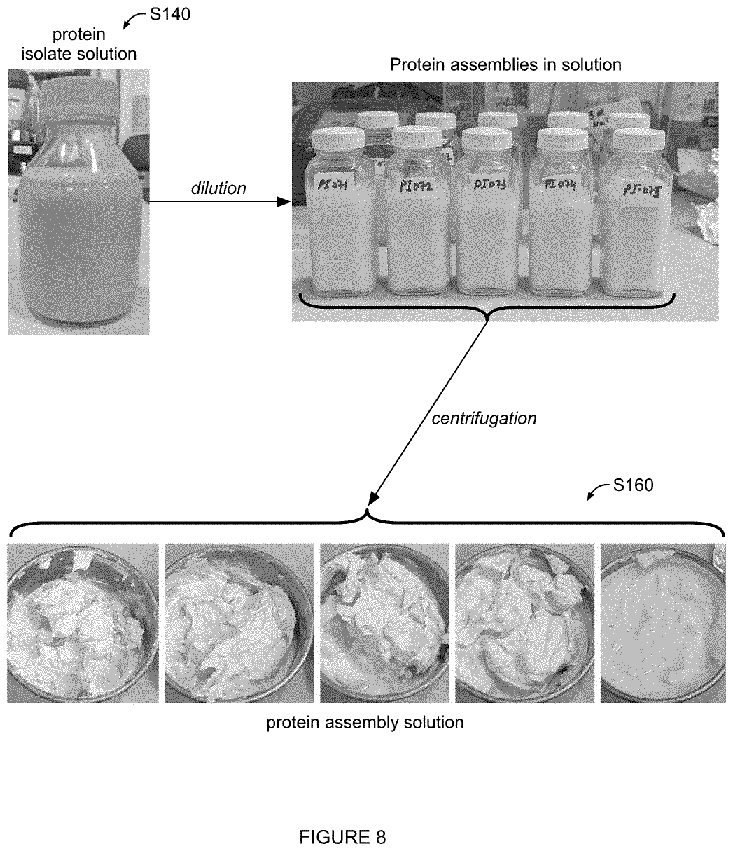

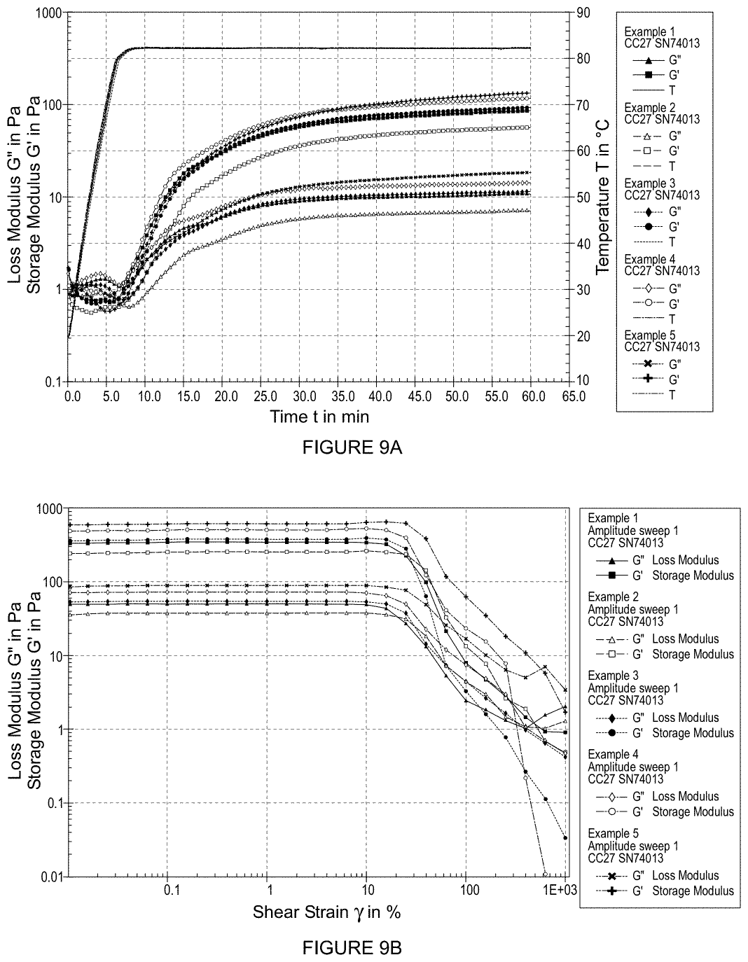

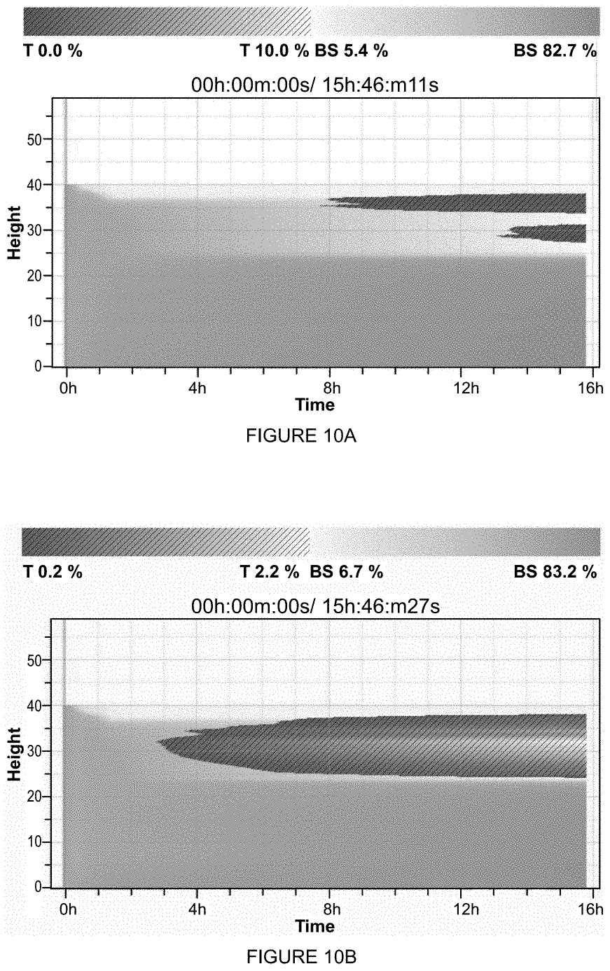

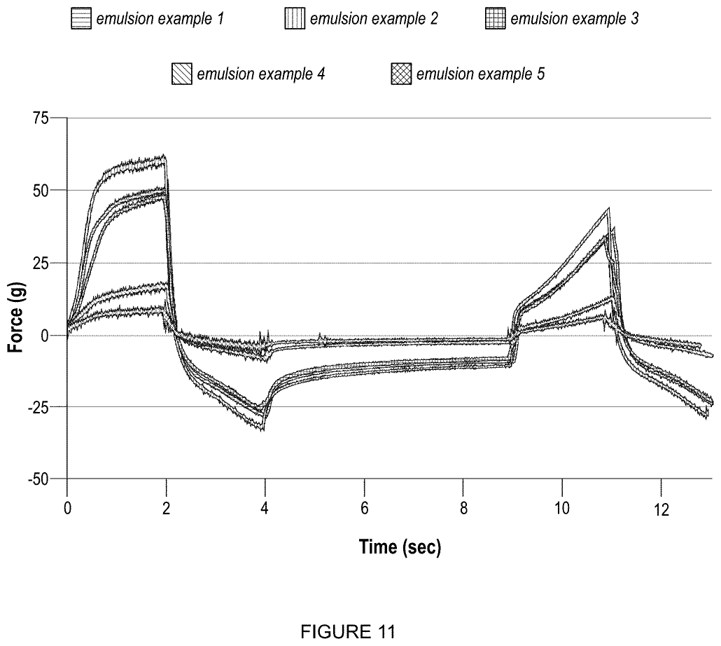

The protein assembly solution (e.g., example shown in FIG. 8) or product produced therefrom (e.g., emulsion, curd, etc.) can be analyzed using any texture analysis technique (e.g., rheology, extension, etc.). In a first example, rheological measurements may measure viscoelastic properties of the protein assembly solution. Viscoelasticity measured by small amplitudes and large amplitude oscillations (LAOS) may characterize a morphology for desired dairy melt, stretch, texture and mouthfeel. Example time sweep rheological data is shown in FIG. 9A; example amplitude sweep rheological data is shown in FIG. 9B. In a second example, a texture profile analysis assay, such as conducted with a texture analyzer, may measure aspects of the texture of a protein curd or derived cheese. A texture profile analysis (“TPA”) test may include two compression cycles on the protein assembly solution or product produced therefrom. The TPA test may quantify stiffness, compression strength, gumminess, springiness, and cohesiveness of the cheese product. Example TPA data is shown in FIG. 11. In a third example, a melt flow assay can be conducted using a rheometer, such that the rheometer may measure the resistance to flow (viscosity) and the changes in cheese morphology/flow as a function of temperature.

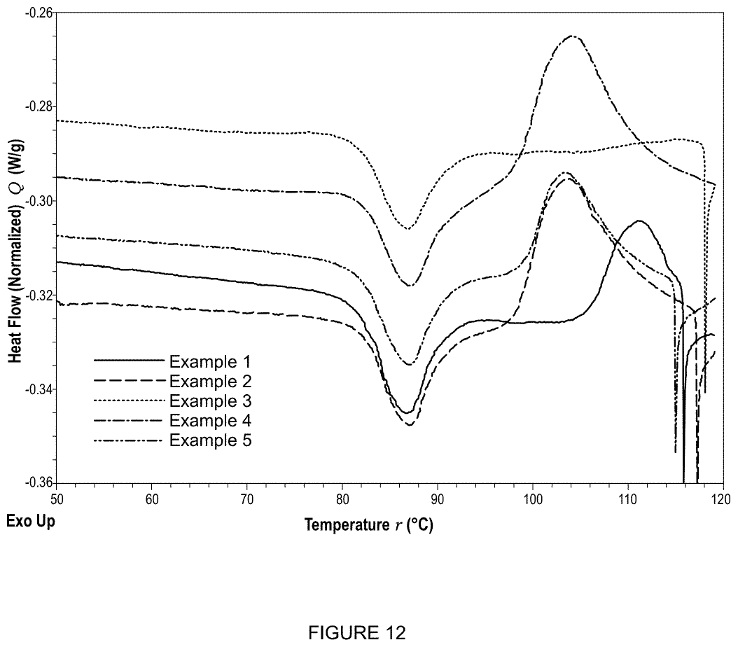

The melt of the protein assembly solution and/or product produced therefrom may be measured by a temperature sweep assay, such as by using a differential scanning calorimeter (“DSC”). The temperature sweep assay may provide a measurement of a temperature at which the protein assembly solution undergoes a thermal phase transition. Additionally, the DSC may quantify the energy of phase transitions and measure onset melting temperature, peak melting temperature, net melting energy, and thermal hysteresis of a melting event. The temperature sweep assay may also indicate a gelation/denaturation point for the protein assembly solution and/or can precisely measure the energy, in the form of heat, of chemical reactions (e.g., namely protein and starch gelation). At elevated temperatures, the DSC may quantify and differentiate between protein denaturation (which demands energy) and gelation (which releases energy). Example DSC data is shown in FIG. 12.

However, any physical parameter of the protein assembly solution and/or product produced therefrom maybe measured (e.g., melt, stretch and toughness, texture, gelation point, color, compression response, water binding capacity, water availability, microstructure, emulsion stability, etc.) using any suitable assay.

However, the protein assemblies can be otherwise formed and/or defined.

The method can include producing an animal product analog S200, which functions to make a plant-based dairy analog and/or any other animal product analog (e.g., meat analog, egg analog, etc.) using the protein extract (e.g., using the protein isolate, using the protein assembly, etc.). The animal product analog can be a vegan product, a food product without animal products and/or with less animal products (e.g., relative to a target animal product), a plant-based food product, a nonmammalian-based food product, and/or any other food product. The animal product analog can optionally be used to manufacture a downstream product. Examples of the animal product analogs and/or downstream products include: milk, cheese, yogurt, cream cheese, dried milk powder, cream, whipped cream, coffee cream, additive ingredients, other dairy replicate products, mammalian meat products (e.g., ground meat, steaks, chops, bones, deli meats, sausages, etc.), fish meat products (e.g., fish steaks, filets, etc.), and/or any other suitable food product. In an example, the animal product analog includes a plant-based milk analog (e.g., a milk analog for cow milk, sheep milk, goat milk, human milk, etc.). In a second example, the animal product analog includes a cheese.

Producing an animal product analog S200 can include: producing a plant-based milk S240; and optionally processing the plant-based milk S260. Alternatively, S200 can include: producing an animal tissue analog using the protein isolate and/or protein assemblies). However, the animal product can be otherwise produced.

Producing the plant-based milk S240 functions to provide a dairy milk substitute that has one or more functional properties similar to that of dairy milk. The plant-based milk is preferably a reconstituted product, but can additionally or alternatively have any other suitable composition.

The plant-based milk can be formed by combining the protein component and additional components (e.g., additional ingredients, such as fatty components, sugars, starches, etc.), and/or otherwise formed.

The protein component is preferably the protein isolates, more preferably the protein assembly, but can additionally or alternatively include the protein units, protein solids (e.g., protein substrate solids and/or particulates; press cake; etc.), and/or any other suitable protein-containing component.

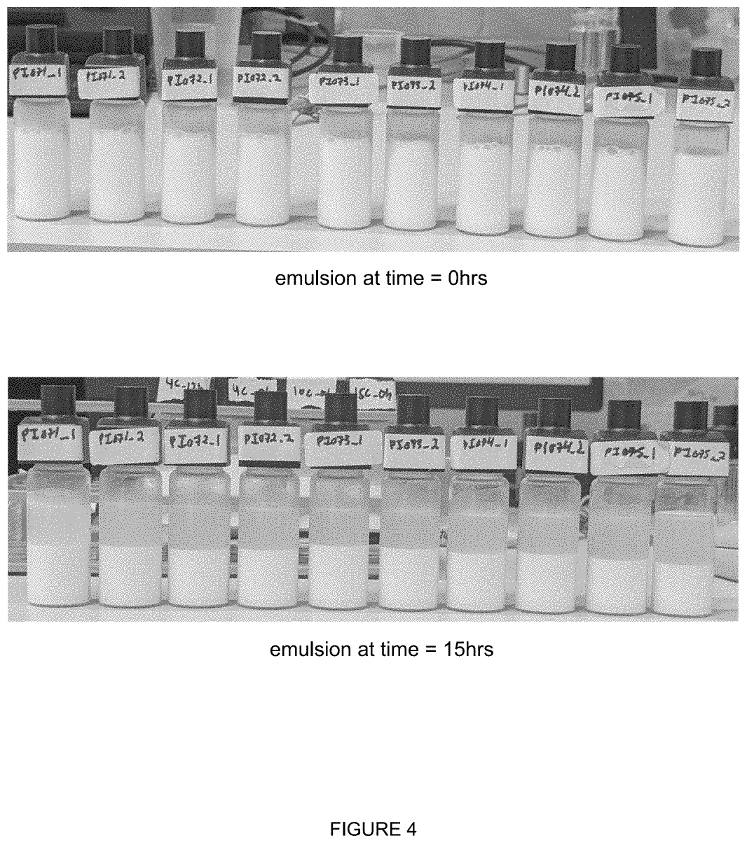

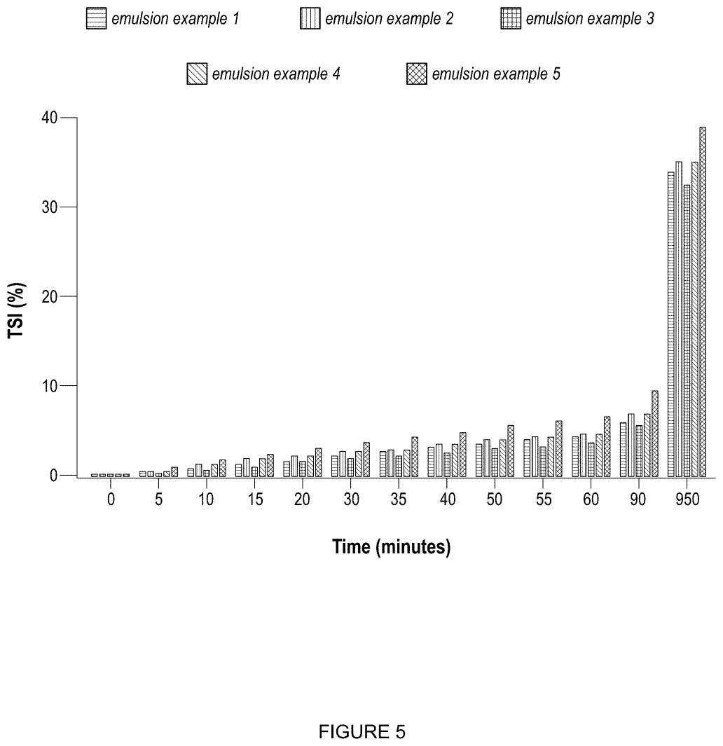

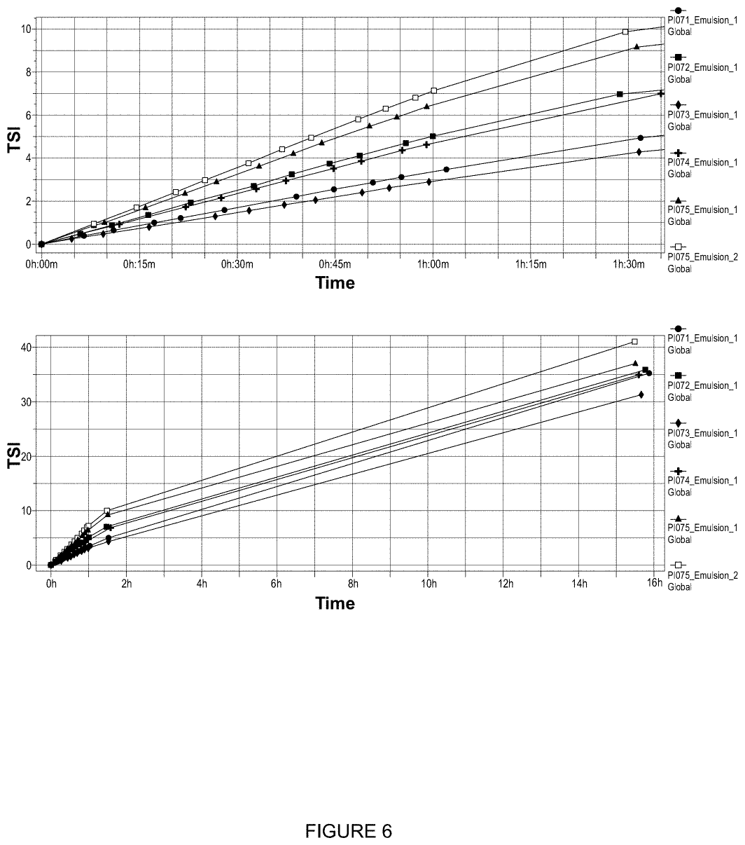

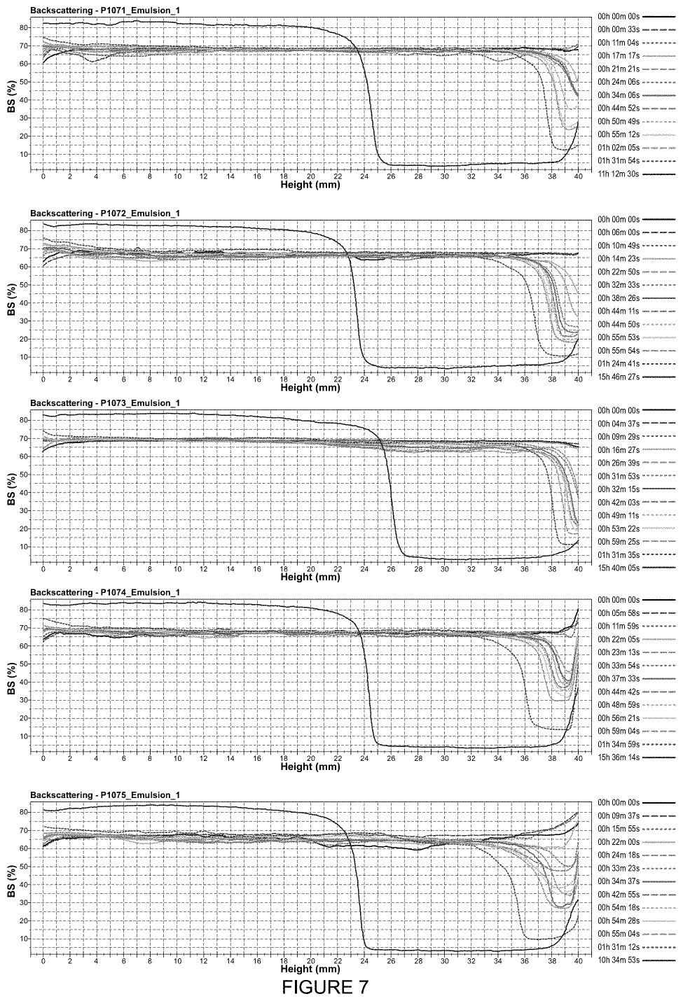

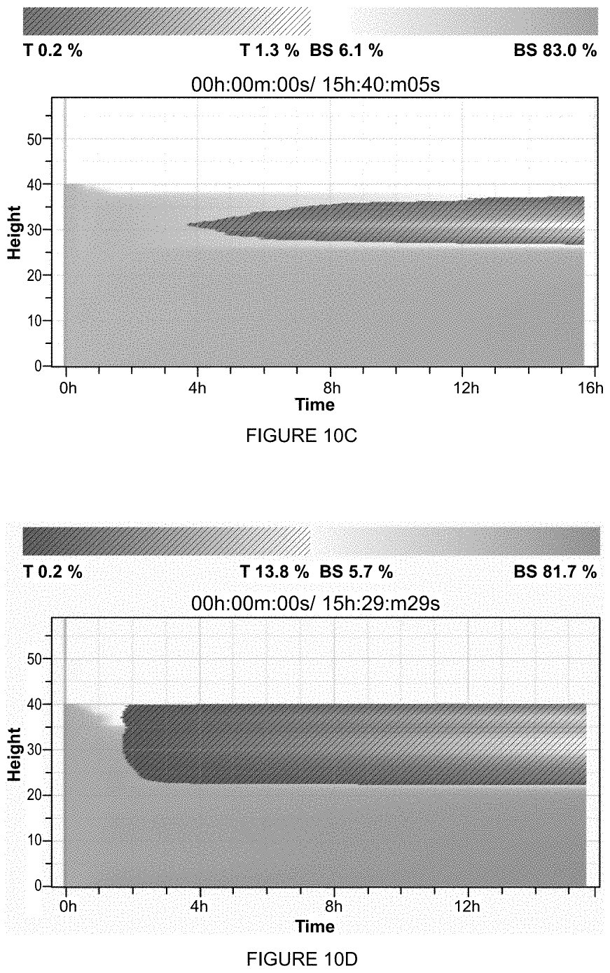

When protein assemblies are used, the protein assemblies can increase a stability of an emulsion between the protein assemblies, an aqueous solution, and a fatty component (e.g., the plant-based milk, a reconstituted product, a separate emulsion, etc.). However, the protein assemblies can decrease the emulsion stability, modify another functional property of the mixture (e.g., melt, stretch, etc.), and/or otherwise affect the mixture. In an example, the emulsion can include a fatty component that is between 1%-50% (e.g., weight per volume, by weight, by volume, by mass, by moles, etc.) or any range or value therebetween (e.g., 2-10%, 4%, etc.), but can alternatively be less than 1% or greater than 50%. The protein assembly solution can include a protein concentration that is between 1%-50% (e.g., by weight, by volume, by mass, by moles, etc.) or any range or value therebetween (e.g., 2-10%, 4%, etc.), but can alternatively be less than 1% or greater than 50%. FIG. 4 depicts an example emulsion at time=0 hrs and at time=15 hrs. A Turbiscan analysis of the emulsion can result in a Turbiscan Stability Index (TSI) that is less than a threshold value for 0-24 hours or any range or value therebetween (e.g., 10 hours, 2 hours, 1 hour, 30 minutes, 20 minutes, 10 minutes, 5 minutes, etc.), but can alternatively the TSI can be less than the threshold value for more than 24 hours. The TSI threshold value can be 1-20 or any range or value therebetween (e.g., 5-15, 10, 5, 2, etc.), but can alternative be less than 1 or greater than 20. During the Turbiscan analysis, the emulsion can be maintained at −80° C. to 1000° C. or any range or value therebetween (e.g., 0° C.-12° C., 4° C., etc.), but can alternatively be maintained at a temperature less than −80° C. or greater than 100° C. Example Turbiscan analysis data is shown in FIG. 5, FIG. 6, FIG. 7, FIG. 10A, FIG. 10B, FIG. 10C, and FIG. 10D.

The additional components can include: fatty components (e.g., fats, oils, etc.), carbohydrates (e.g., sugars, starches, fibers, polysaccharides, such as maltodextrin, gums, etc.), enzymes (e.g., transglutaminase, tyrosinase, bromelain, papain, ficain, other cysteine endopeptidases, etc.), emulsifiers (e.g., lecithin), hydrocolloids (e.g., thickening agents, gelling agents, emulsifying agents, stabilizers, etc, such as starch, gelatin, pectin, and gums, such as agar, alginic acid, sodium alginate, guar gum, beta-glucan, xanthan gum, etc.), salts (e.g., NaOH, NaCl, CaCl₂), etc.), and/or any other component.

The reconstituted product can be used as a food product, as an intermediate product used in food manufacture (e.g., manufacture of a plant-based food product), and/or otherwise used.

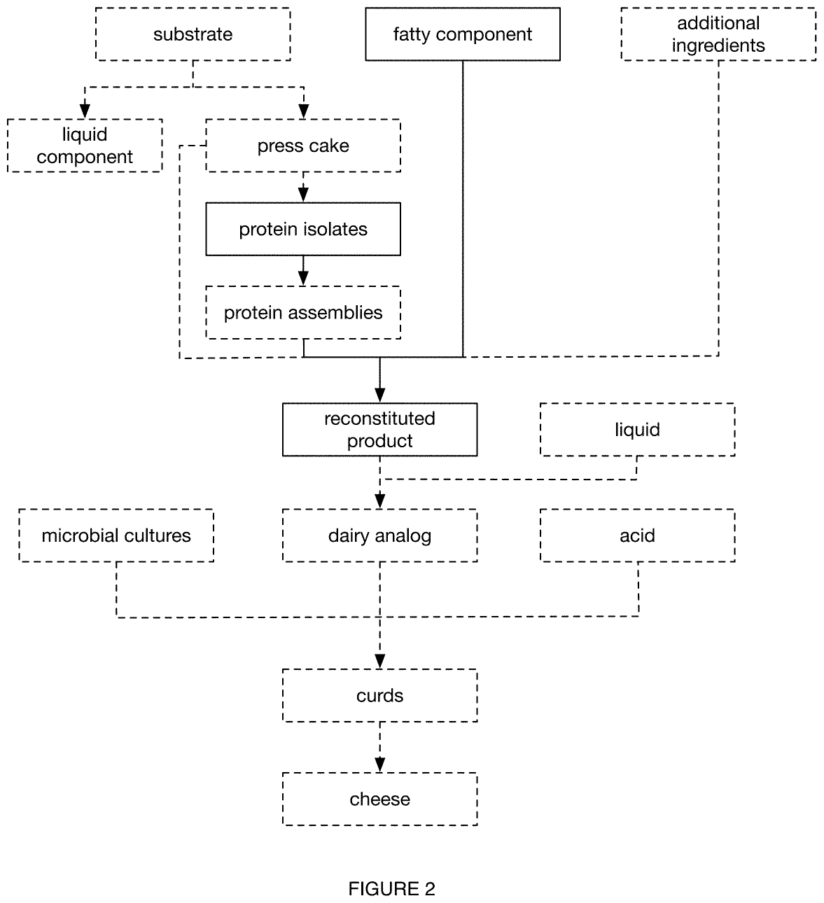

Forming a reconstituted product can include combining: the protein assemblies (e.g., the protein assemblies in solution), a fatty component, a substrate and/or component thereof (e.g., the same substrate used to produce the protein assemblies or a different substrate; the same press cake used to produce the protein assemblies or a different press cake; etc.), additional ingredients, and/or any other component. S240 can be performed after S160, during S200, as a separate process from animal product analog manufacture (e.g., separate from S200), during and/or after S260, and/or at any other time. An example is shown in FIG. 2.

The milk analog (e.g., reconstituted product) can be: a suspension, a colloid, an emulsion, a homogeneous mixture, a gel, a solid, and/or have any other suitable structure or form factor. In an example, the milk analog can be produced by emulsifying the protein component (e.g., protein isolate and/or protein assemblies) with a lipid component. In another example, the milk analog can be produced by emulsifying the protein component (e.g., protein isolate and/or protein assemblies) and a lipid component within an aqueous continuous phase. However, the milk analog can be otherwise produced.

Reconstituted product components may be selected based on desirable and/or complimentary attributes, such as: cost, nutritional content, flavoring, functionalities, appearance, allergenicity, climate impact (e.g., carbon impact), and/or any other attribute. The reconstituted product components can be selected based on the respective attribute values (e.g., the fatty component with the highest attribute value(s) is selected), based on an optimization (e.g., between nutrition, cost, extraction difficulty, etc.), and/or based on a collective attribute profile (e.g., a combination of fats selected to produce a desired nutritional profile).

The reconstituted product components and/or amounts thereof are preferably selected such that the resultant product (e.g., the reconstituted product, a downstream product formed using the reconstituted product, etc.) has attributes that substantially match one or more attributes (e.g., texture, flavor, macronutrient profiles, amino acid profiles, etc.) of a target product (e.g., bovine milk). In a first example, fatty components can be selected such that the fatty components collectively match that of milk and/or any other dairy product. In a second example, the components and amounts thereof can be selected such that a nutritional profile (e.g., fat profile) of the reconstituted product substantially matches that of milk and/or any other dairy product. In a specific example, when the protein assemblies, fatty component, and/or additional ingredients in the reconstituted product are impure, and/or include other macronutrients, components can be selected to compensate for the impurities such that the overall reconstituted product (or downstream product therefrom) matches the nutritional profile of a target product. In a third example, components can be selected to exclude: allergenic ingredients (e.g., peanut, tree nuts, etc.), soy, canola ingredients (e.g., canola oils), gums, hydrocolloids, saturated fats, and/or any other component. In a specific example, components can be selected such that the reconstituted product contains no or less than a threshold percent (e.g., 0.25%, 0.5%, 1%, 2%, 5%, any range therein, a percentage greater than 5%, a percentage less than 0.25%, etc.) allergenic ingredients, canola ingredients, gums, hydrocolloids, and/or any other ingredient.

The fatty component is preferably selected such that it substantially matches one or more fat profile parameters of bovine milk, but can alternatively substantially match another target fat profile. The fat profile parameters can include: types of fats (e.g., saturated fat, unsaturated fats, etc.), chain lengths, percent by weight, percent by mol, and/or any other nutritional descriptor. In an illustrative example the fatty component can include 60-70% saturated, 20-30% monounsaturated, and 1-5% polyunsaturated fatty acids (e.g., with oleic acid the predominate monounsaturated fatty acid, with 11% of the saturated fatty acids including short-chain fatty acids, etc.). The fatty component is preferably derived from different sources from that of the protein assembly (e.g., a different plant substrate, a different instance of a press cake used to make the protein assembly, etc.), but can additionally or alternatively be from the same source (e.g., the same plant substrate, the same press cake, etc.). The fatty component could be refined, clarified, fractionated, and/or otherwise processed to optimize the lipid profile (e.g., for a target end product functional parameter profile). The fatty component sources are preferably plants (e.g., reproductive organs, leaves, stems, etc.), but can additionally or alternatively be animals and/or any other source.

The desirable attributes of the fatty component may be selected based on compatibility with attributes of the protein assembly and/or additional reconstituted product components, based on nutritional concerns, based on similarity to a target food product, and/or otherwise selected. In a first embodiment, a fatty component may be selected to have a “pleasing” taste that masks an “unpleasing” taste present in another reconstituted product component (e.g., the protein assembly, a press cake, etc.). In a second embodiment, a fatty component may be selected to have a desirable nutritional content. In an illustrative example, the fatty component can be selected to have lower saturated fat content than bovine milk, but recovers desirable textural properties through treated unsaturated fats. In a third embodiment, a fatty component may be selected to have a desired crystalline structure, phase change profile (e.g., melting profile, solid at room temperature, etc.), and/or otherwise selected. In a first example, certain usages of the reconstituted product (e.g., to manufacture a food product) may necessitate that the reconstituted product include sufficient saturated fat content. For instance, it may be desirable for a cheese analog produced from a reconstituted product to have a sufficiently high saturated fat content to achieve a melting behavior as a function of temperature that is similar to a dairy cheese. Other reconstituted product components (e.g., the protein assembly, a press cake, etc.) may be made from one or more substrates that are low in saturated fat and/or may otherwise have lost saturated fats (e.g., a press cake may have lost saturated fats during the process of producing the press cake). Therefore, a fatty component maybe selected that is high in saturated fat (e.g., more than 50%, 60%, 70%, 80%, or 90%; a range therebetween; etc.), such as coconut oil or palm oil. In a second example, a press cake may be made from a substrate that includes polyunsaturated fatty acids. The polyunsaturated fatty acids may optionally be removed during the extraction process. In a third example, a fatty component may be selected that does not include polyunsaturated fatty acids.

The fatty component is preferably a triglyceride, but can alternatively be a monoglyceride, diglyceride, phospholipid, and/or any other lipid. The triglyceride can be saturated, unsaturated (e.g., monounsaturated, polyunsaturated, etc.), and/or have any other classification. The fatty component is preferably be derived from one or more plant sources, but can alternatively be derived from animal sources (e.g., dairy butter, lard, tallow, insect fats, etc.) and/or any other source. Examples of fatty components include: avocado oil, mustard oil, coconut oil, palm oil, peanut oil, canola oil, cocoa butter, grapeseed oil, olive oil, rice bran oil, safflower oil, sesame oil, sunflower oil, soybean oil, pumpkin seed oil, kokum butter, shea butter, mango butter, hemp oil, vegetable oil, any other neutral lipid, a synthetic lipid, any combination thereof, no or less than a threshold percentage of a lipid type (e.g., canola oil), and/or any other lipid. The fatty component may include leftover lipids from the formation of a press cake (e.g., extracted from a substrate). The fatty component can be obtained from the same substrate from which the proteins were extracted or be obtained from a different substrate. The fatty component may include a combination of fatty components. In a first example, a blend of lipids that are solid at room temperature (e.g., fats) and lipids that are liquid at room temperature (e.g., oils) can be used. In a second example a blend of different varieties of plant-based oils, a blend of different varieties of animal-based oils, and/or a blend of plant- and animal-based oils may be used. Optionally, the fatty component can be selected and/or processed (e.g., adjusting saturation, lipid crystalline structure, melting point, smoke point, etc.) such that the fatty component has a target melt property profile (e.g., solid at room temperature, liquid at room temperature, matching a dairy melt property profile, etc.).

A percentage (e.g., by mass, by weight, by moles, by volume, etc.) of the fatty component within the reconstituted product can be between 1%-95% or any range or value therebetween (e.g., 10%-65%, 7%-45%, 2%-5%, 3%-4%, etc.), but can alternatively be less than 1% or greater than 95%. A percentage (e.g., by mass, by weight, by moles, etc.) of the protein assemblies and/or total protein content (e.g., wherein the total protein content includes a combination of protein assemblies and other protein components in the reconstituted product) within the reconstituted product can be between 2%-50% or any range or value therebetween (e.g., 5%-40%, 5%-10%, 20%-40%, etc.), but can alternatively be less than 2% or greater than 50%. The percentage (e.g., by mass, by weight, by moles, etc.) of protein assemblies within the total protein content (e.g., where 100% is equivalent to all proteins in the reconstituted product deriving from the protein assembly solution) can be between 1%-100% or any range or value therebetween (e.g., 25%-75%, 75%-100%, etc.), but can alternatively be less than 1%.

The ratio of the fatty component to protein (e.g., protein assemblies and/or total protein content) may be selected depending on the desired application of the reconstituted product. For instance, using the reconstituted product to produce a cheese analog may entail using a higher ratio of protein-to-fatty component than using the reconstituted product to produce, for instance, a butter analog. In a first example, a range of 0.05 to 10 (e.g., 0.2 to 5; excluding ratios less than 0.1 and/or greater than 7; etc.) for the ratio of protein-to-fatty component by mass may be considered depending on the desired attributes for the reconstituted product. In a second example, a range of 0.5 to 1.5 (e.g., a range of 0.7 to 1, excluding ratios less than 0.3 and/or greater than 2, etc.) for the ratio of proteins to fats by weight may be considered depending on the desired attributes for the reconstituted product. However, any other ratio can be achieved.

The additional ingredients are preferably food-safe, but can alternatively be not food safe (e.g., wherein ingredients that are not food safe can be removed in a downstream process). Examples of additional ingredients that can be used as reconstituted product components include: coloring, enzymes (e.g., rennet enzymes), mold powders, microbial cultures, particulates, water (e.g., for dilution), macronutrients (e.g., protein, fat, starch, sugar, etc.), gums, hydrocolloids, flavoring compounds, nutrients, micronutrients, chemical crosslinkers and/or non-crosslinkers (e.g., L-cysteine), proline-rich proteins (e.g., gluten), a substrate and/or component therefrom (e.g., a press cake), salts, acids, bases, emulsifiers (e.g., to increase colloidal stability), carbon sources (e.g., to supplement fermentation), no or less than threshold percentage (e.g., a maximum percentage, such as 1% by weight) of gums or hydrocolloids, any combination thereof, and/or any other ingredient.

In a first example, the additional ingredients are selected to bring the reconstituted product (or a downstream product produced from the reconstituted product) within an acceptable range of an attribute of an animal product.

In a second example, the additional ingredients are selected such that a nutritional profile (e.g., macronutrient profile) of a product produced from the reconstituted product substantially matches a target nutritional profile (e.g., for a target dairy product). The macronutrients in the reconstituted product are preferably the same macronutrients as those contained in milk, but alternatively can be different macronutrients. The macronutrient characteristics (e.g., chain length, functionality, amino acid profile, etc.) in the product are preferably the same characteristics as those for milk macronutrients, but alternatively can be different characteristics.

In a first illustrative example, additional ingredients are selected such that at least 7000 mL/g of sugar is present in a milk analog formed from the reconstituted product.

In a second illustrative example, ingredients are selected such that approximately 4-6% contains carbohydrates, 2-4% contains fat, and 3-4% contains protein (e.g., by weight).

In a third illustrative example, ingredients are selected such that the milk analog formed from the reconstituted product is a milk analog for human milk (e.g., containing approximately 0.5 to 1.5 g/dL for total protein, 1.5 to 10 g/dL for fat, and 5 to 8 g/dL for carbohydrates).

The protein assembly, fatty component, and/or any additional components may be combined by mechanical means and/or any other combination method. For example, the components can be combined using any comminution, mixing, emulsification, and/or homogenization method. Examples of comminution methods include: pulverizing, blending, crushing, tumbling, crumbling, atomizing, shaving, grinding, milling, cryo-milling, chopping, and/or any other method. Homogenization (e.g., to form an emulsion between the protein assembly solution and the fatty component) can include using a rotor stator system and/or any other homogenizer device. The resultant emulsion can have an aqueous continuous phase with fatty droplets, and/or be otherwise structured. When protein isolates and/or protein assemblies are used, the protein isolates and/or protein assemblies are preferably primarily in the aqueous continuous phase (e.g., more than 50%, 60-%, 70%, 80%. 90%, etc. of the protein isolates and/or protein assemblies are in the aqueous phase), but can alternatively be primarily in the fatty droplets. The homogenizer device preferably imparts a shear force below a threshold (e.g., to not break up protein structures in the protein solution), but can alternatively impart any shear force. In an example, a rotor stator system can used at an rpm between 1,000 rpm-50,000 rpm or any range or value therebetween (e.g., 5,000 rpm-15,000 rpm, 10,000 rpm, etc.), but can alternatively be used at less than 1,000 rpm or greater than 50,000 rpm. The rotor stator system can used for 10 seconds-1 hour or any range or value therebetween (e.g., 30 seconds-5 minutes, 1 minute, etc.), but can alternatively be used for less than 10 seconds or greater than 1 hour. However, a rotor station system can be used at any other rpm and for any amount of time. The reconstituted product can be a slurry, cake, liquid, viscous liquid, emulsion, suspension, precipitate, and/or any other product.

Components can be combined concurrently and/or asynchronously. In a first example, the protein assembly solution and the fatty component are homogenized to form an emulsion, wherein additional ingredients are subsequently added to the emulsion (e.g., such that the resulting reconstituted product can function as a milk analog). In a second example, a press cake may be soaked in a liquid (e.g., water, salt water, etc.), wherein an industrial homogenizer may be used to blend the soaked press cake with the fatty component. The protein assemblies may then be added to the homogenized mixture using a second homogenization step. In a fourth example, the protein assembly solution, the fatty component, and/or additional ingredients are concurrently homogenized using a single homogenization step to form the reconstituted product. Optionally, the fatty component can be heated prior to and/or during combination. Heating the fatty component can optionally transition the fatty component from a solid to a liquid, which can facilitate homogenization. The fatty component is preferably heated below a threshold temperature (e.g., 85° C.) such that proteins in the protein assembly solution do not denature, but can alternatively be heated to any temperature. For example, the fatty component can be heated to a temperature between 25° C.-100° C. or any range or value therebetween (e.g., less than 85° C., 30-50° C., 40° C., etc.), but can alternatively be less than 25° C. or greater than 100° C.

In variants, the plant-based milk can be manufactured by producing the reconstituted product, then diluting the reconstituted product with a liquid or other thinner. This can be particularly useful when the reconstituted product is gel-like or has a viscosity higher than a predetermined threshold (e.g., more than 2.0 cp, 1.0 cp, etc.; more than 1.02-1.05 specific gravity at 16° C., etc.), which can result when a fat (e.g., solid at room temperature) and/or the protein assemblies are used; and/or when other conditions are met. Diluting the reconstituted product can include: combining the reconstituted product with a liquid (e.g., producing a slurry from the reconstituted product, etc.). The liquid can be water, a salt solution, a sodium hydroxide solution, and/or any other liquid. The ratio of reconstituted product-to-liquid may be variable depending on the purposes of the animal product analog, such that a higher ratio of reconstituted product-to-liquid may produce a more viscous dairy analog and a lower ratio of reconstituted product-to-liquid may produce a less viscous dairy analog. The mixture of the reconstituted product and liquid may be homogenized, mixed, comminuted (e.g., until the mixture has reached the desired consistency of the slurry), and/or otherwise combined. Examples of comminution methods include: pulverizing, blending, crushing, tumbling, crumbling, atomizing, shaving, grinding, milling, cryo-milling, chopping, and/or any other method. Any suitable equipment for carrying out any of these processes can also be used. In some embodiments, at least blending is used. For example, the mixture may be blended until the dairy analog (e.g., the slurry) is substantially in liquid form, although there may still be particulates remaining. Solid masses may be filtered or separated out by any of known means, leaving the remaining liquid as a milk analog. Alternatively, or additionally, the solid masses may be further blended, the dairy analog may be further blended, the solid masses may be left in the dairy analog, and/or solid masses may not be present in the dairy analog (e.g., when the reconstituted product does not include solid masses). An aspect of the present disclosure may be directed towards a milk analog produced from a reconstituted product, such as the milk analog as produced by the preceding process.

However, the plant-based milk can be otherwise formed.

The method can optionally include processing the plant-based milk S260, which functions to: produce a downstream animal product analog (e.g., a plant-based cheese analog) and/or an intermediate product to the downstream product (e.g., a cheese curd analog), to prime the plant-based milk for making the downstream product, to preserve the plant-based milk, and/or for any other purpose. Processing the animal product analog can include: adjusting the pH level, adjusting the temperature, adding microbial cultures and/or other ingredients, emulsifying the animal product analog, curdling (e.g., gelling) the animal product analog, obtaining curds from the animal product analog, processing curds from the animal product analog, and/or any other processing step. Processing methods can be performed in any order and performed any number of times.

In a specific example, the animal product analog can be processed to produce a cheese in the same way a dairy product (e.g., non-analog product) would be processed to produce a cheese. For example, additional ingredients (added to the plant milk during S260) can be selected to be the same as or as close to the target animal product. For example, blue cheese microbial cultures that are used to make dairy-based blue cheese can be selected for inclusion in the reconstituted product (e.g., to make a plant-based blue cheese analog). In another example, all ingredients and processes can be the same as dairy-based food product manufacture, except that the dairy milk is replaced with the reconstituted product (e.g., the plant milk). However, the additional ingredients and/or process steps can be otherwise selected.

Adjusting the pH level of the animal product analog can include determining an initial pH (e.g., via measuring, calculating and/or estimating based on dairy analog formulation, etc.), comparing the pH to a target pH, and adjusting the pH based on the comparison. This can function to induce curdling in the plant-based milk, to prepare the plant-based milk for downstream processes (e.g., create a more favorable environment for inoculation with a microbial culture), and/or performed for other purposes. In a first variant, adjusting the pH includes raising pH (e.g., prior to curdling). In an example, the pH can be raised to a target pH (e.g., 7 to 14, 8 to 12, 9 to 10, not lower than 6, not higher than 14, etc.) by adding one or more bases (e.g., a solution containing sodium hydroxide). In a second variant, adjusting the pH includes lowering the pH (e.g., to induce curdling). In an example, the pH can be lowered to a target curdling pH (e.g., 3.5 to 5.5, 4 to 5, 4.5 to 4.75, not lower than 3, not higher than 7, etc.) by adding one or more acids (e.g., a solution containing sodium hydroxide). In a third variant, adjusting the pH includes raising the pH and lowering the pH (e.g., in any order). However, the pH level of the diary analog can be otherwise adjusted.

Adjusting the temperature of the animal product analog preferably includes heating the animal product analog (e.g., to the denaturation point of the protein isolates and/or protein aggregates, to under the denaturation point, etc.), but can alternatively include cooling the animal product analog (e.g., heating followed by cooling, blanching, etc.). The adjusted temperature and/or the amount of time the adjusted temperature is maintained can be selected based on a microbial culture (e.g., a culture already added to the dairy analog, a culture which will be added to the dairy analog, etc.). In examples, the adjusted temperature can be: 0-200 degrees Fahrenheit, 85-120 degrees Fahrenheit, 100-130 degrees Fahrenheit, 72 degrees Fahrenheit, 65-75 degrees Fahrenheit, above 25° C., 30° C., 40° C., 50° C., and/or above any other temperature, below 25° C., 30° C., 40° C. (e.g., below 32° C.), 50° C., and/or below any other temperature, and/or be any other temperature. However, the temperature of the diary analog can be otherwise adjusted. The temperature of the animal product analog can be adjusted and/or held: after pH adjustment, during and/or after inoculation (e.g., held in a bath for the inoculation time period), and/or at any other point in time.

Adding microbial cultures to the animal product analog (e.g., the dairy analog) can optionally include steeping the animal product analog in one or more cultures (e.g., where the animal product analog rests at a target temperature after the cultures are added). The cultures can include cultures for cheeses such as blue, camembert, cheddar, alpine, parmesan, swiss, and/or any other microbial culture and/or combination thereof. Examples of microbial cultures that can be used include: cheese cultures (e.g., cheese starter cultures), yogurt cultures, wine cultures, beer cultures, and/or any other microbial culture. Examples of cultures that can be used include: Arthrobacter arilaitensis, Arthrobacter bergerei, Arthrobacter globiformis, Arthrobacter nicotianae, Arthrobacter variabilis, Bifidobacterium adolescentis, Bifidobacterium animalis, Bifidobacterium bifidum, Bifidobacterium breve, Bifidobacterium infantis, Bifidobacterium lactis, Bifidobacterium longum, Bifidobacterium pseudolongum, Bifidobacterium thermophilum, Brachybacterium alimentarium, Brachybacterium tyrofermentans, Brevibacterium aurantiacum, Brevibacterium casei, Brevibacterium linens, Candida colliculosa, Candida kefyr, Candida jefer, Candida krusei, Candida mycoderma, Candida utilis, Candida vini, Candida zeylanoides, Carnobacterium divergens, Carnobactrium maltaromaticum, Corynebacterium ammoniagenes, Corynebacterium casei, Corynebacterium flavescens, Corynebacterium mooreparkense, Corynebacterium variabile, Cystofilobasidium infirmominiatum, Debaryomyces hansenii, Debaryomyces kloeckeri, Enterococcus faecalis, Fusarium domesticum, Geotrichum candidum, Hafnia alvei, Halomonas, Issatchenkia orientalis, Kazachstania exigua, Kazachstania unispora, Kluyveromyces lactis, Kluyveromyces marxianus, Kocuria rhizophila, Kocuria varians, Lactobacillus acidipiscis, Lactobacillus acidophilus, Lactobacillus brevis, Lactobacillus bulgaricus, Lactobacillus casei, Lactobacillus corynmformis, Lactobacillus curvatus, Lactobacillus delbrueckii, Lactobacilus fermentum, Lactobacillus gasseri, Lactobacillus johnsonii, Lactobacillus kefiranofaciens, Lactobacillus kefiri, Lactobacillus nodensis, Lactobacillus parabrevis, Lactobacillus paracasei, Lactobacillus parakefiri, Lactobacillus paraplantarum, Lactobacillus pentosus, Lactobacillus perolents, Lactobacillus planarum, Lactobacillus rhamnosus, Lactobacillus salivarius, Lactobacillus tucceti, Lactococcus lactis, Lactococcus raffinolactis, Lecanicillium lecanii, Leuconostoc citreum, Leuconostoc citovorum, Leuconostoc dextranicum, Leuconostoc pseudomesenteroides, Leuconostoc kimchi, Leuconostoc mesenteroides, Macrococcus caseolyticus, Microbacterium foliorum, Microbacterium gubbeenense, Micrococcus luteus, Pediococcus, Penicillium album, Penicillium camemberti, Penicillium caseifulvum, Penicillium chrysogenum, Penicillium commune, Penicillium nalgiovense, Penicillium roqueforti, Pichia fermentans, Propionibacterium acidipropionici, Propionibacterium freudenreichii, Propionibacterium jensenii, Proteus vulgaris, Psychrobacter celer, Rhodosporidium infirmominiatum, Rhodotorula minuta, Saccharomyces cerevisiae, Staphylococcus carnosus, Staphylococcus equorum, Staphylococcus fieurettii, Staphylococcus saphrophyticus, Staphylococcus sciuri carnaticus, Staphylococcus succinus, Staphylococcus vitulinus, Staphylococcus xylosus, Streptococcus cremoris, Streptococcus lactis, Streptococcus lactis subspecies diacetylactis, Streptococcus thermophilus, Streptococcus gallolyticus, Streptococcus salivarius, Thrichosporon beigelii, Verticillium lecanii, Yarrowia lipolytica, Zygotorulaspora florentina, the genuses thereof, the families thereof, the phyla thereof, and/or any other suitable microbe and/or combination thereof. The steeping time can be 30 minutes to 2.5 hours and/or any other steeping time. Steeping can be terminated when the dairy analog has thickened above a threshold (e.g., as determined by a viscosity measurement, viscoelastic property analysis, etc.).

Curdling the animal product analog (e.g., the dairy analog) is preferably performed after microbial culture addition, but can alternatively be performed at any other time. Curds can be any substantially solid and/or semi-solid formation (e.g., a gel). Curds can be formed by: acid-induced curdling (e.g., acidification), salt-induced curdling, heating, fermentation, emulsification, and/or using any other method. In a first variant, curdling the dairy analog can include adding a salt mix to the dairy analog. In an illustrative example, a solution including approximately 0.5 g of CaCl₂) and approximately 3 g MgCl₂ may be combined with approximately 5 mL of water to create the salt mix. In a second variant, curdling the dairy analog can include adding an acid mix to the dairy analog. In an illustrative example, a ratio of approximately 2 g of citric acid may be added to 5 mL of water to create the acid mix. In a third variant, curdling the dairy analog can include adding enzymes to the dairy analog. In an example, the enzymes that can be used include: transglutaminase, chymosin, tyrosinase, rennet enzymes and/or rennet-type enzymes (e.g., from animal sources, plant-based sources, microbial sources, etc.), and/or any other suitable enzyme. In a fourth variant, curdling the dairy analog can include adjusting the temperature of the dairy analog (e.g., heating the dairy analog to a target temperature for a target period of time). In a fifth variant, curdling the dairy analog can include any combination of adding salt, adding acid, adding enzymes, adjusting the temperature, and/or any other processing step (e.g., in any order). However, curdling the dairy analog can be otherwise performed. The acidified mixture can optionally be incubated at above room temperature, above 30° C. above 40° C., above 50° C., above, below, at, or near the denaturation point of the protein, at a temperature favorable to the microbial culture, and/or at any other suitable temperature.

Obtaining curds from the dairy analog (e.g., isolating curds, collecting curds, etc.) can function to isolate the curds for further processing. This preferably occurs after the dairy analog curdles to form a substantially solid portion (e.g., curds) and a substantially liquid portion (e.g., whey), but can alternatively be performed at any other time. Separating the curds can include pouring the dairy analog through a filter and/or strain to capture the curds. However, separating the curds can be otherwise performed.

Processing curds from the dairy analog can function to produce a cheese analog, a dairy food product analog, and/or any other food product. Processing the curds can be performed after separating the curds from the dairy analog, prior to separating the curds from the dairy analog, and/or at any other time. Processing can include: pressing the curds (e.g., to a target moisture content), cutting the curds, cheddaring, salting the curds, adjusting the temperature of the curds (e.g., incubation at a target temperature and for target period of time), shaping the curds, aging the curds, and/or any other processing method. Cutting the curds can be performed prior to separating the curds from the dairy analog (e.g., to increase further whey drainage from the curds) and/or after separating the curds from the dairy analog. Aging (e.g., including ripening, fermenting, affinage, etc.) can be for a target period of time (e.g., optimal aging time for a target flavor profile, texture profile, etc.) and/or at one or more target temperatures (e.g., optimal for microbes in the curds). However, the curds can be otherwise processed.

However, the dairy analog can be otherwise processed.

A first example includes an animal product analog made from one or more ingredients extracted from a waste product of edible oil production (e.g., a press cake; an oilseed press cake; etc.). In this example, the waste product can be used without an additional lipid extraction step before use as an ingredient in a plant-based food; alternatively, the waste product can be additionally post-processed (e.g., to remove remaining fats, off-flavors, etc.). Examples of ingredients that can be derived from the waste product include: proteins, polysaccharides, flavors, small molecules, carbohydrates, waste product solids (e.g., powders, fibers, etc.), other macromolecules (e.g., cellulose, starch, nucleic acids, etc.), macronutrients, micronutrients, and/or any other suitable ingredient or derivative product. Examples of the animal product analog that can be made include: milk, dairy products (e.g., cheese, cream cheese, yogurt, etc.), tissue replicas (e.g., wherein the proteins can be formed into a matrix by gelling, crosslinking, or otherwise treated), egg products (e.g., powdered egg, scrambled egg, etc.), and/or any other suitable animal product analog. In variants, the protein extract can be assembled into micelles, wherein the micelles are used as an ingredient in the plant-based dairy analog. Alternatively, the protein extract can be used without higher-order protein assembly (e.g., agglomeration, aggregation, micellarization, etc.). The micelles can be higher-order (e.g., quinary) protein structures formed from multiple protein oligomers (e.g., globulin hexamers; 11S globulin hexamers; etc.), or have any other structure (e.g., include a lipid exterior, encapsulating proteins therein; etc.). The protein oligomers can self-aggregate into the micellar structure under certain conditions (e.g., rapid dilution), and/or be otherwise created. In an example, the protein micelle can have a diameter between 50 nm and 800 nm (e.g., between 100 nm and 300 nm).

A second example includes a plant-based milk made from protein assemblies (e.g., micelles; micellar protein isolates (MPIs)). The protein assemblies can be substantially pure protein (e.g., exclude substantial amounts of lipids, salts, etc.), and optionally include an aqueous solution. The protein assemblies can be higher-order (e.g., quinary) protein structures formed from multiple protein oligomers (e.g., globulin hexamers; 11S globulin hexamers; etc.; wherein the protein oligomers can self-aggregate into the protein structure; etc.), or have any other structure (e.g., include a lipid exterior, encapsulating proteins therein; etc.). In variants, the protein assemblies can be mixed (e.g., emulsified) with a lipid component, wherein the lipid component is in the dispersed phase and an aqueous solution is in the continuous phase; the protein assemblies are preferably within the aqueous solution/continuous phase (e.g., primarily), but can additionally or alternatively be within the lipid component. The plant-based milk can additionally or alternatively include other ingredients, such as plant solids (e.g., protein powder, fiber, etc.), salts, sugars, and/or other ingredients.

A third example includes a plant-based dairy product made from protein assemblies (e.g., micelles; micellar protein isolates). The plant-based dairy product is preferably manufactured using the plant-based milk described in the second illustrative example, but can additionally or alternatively use any other suitable ingredient. In an example, the plant-based dairy product is manufactured using one or more standard dairy product manufacturing techniques (e.g., the entirety of a standard dairy manufacturing process, one or more steps from a standard dairy manufacturing process, etc.), while replacing dairy milk with the plant-based milk. In a second example, the plant-based dairy product is manufactured using the plant-based milk, using different techniques to achieve comparable intermediary and/or final products (e.g., comparable as measured by similarity across a predetermined set of functional properties). In an illustrative example, when making a plant-based cheese, the plant-based milk can be cultured (e.g., inoculated) with microbial cultures before heating and/or curdling. In a second illustrative example, when making a plant-based cheese, curdling in the plant-based milk can be induced by acidification (e.g., using salts and/or acids) instead of or in addition to heating.

However, the plant-based food product can be otherwise made with any other set of ingredients, and have any other composition.

Unless otherwise indicated, all numbers and expressions, such as those expressing dimensions, physical characteristics, etc., used in the specification (other than the claims) are understood as modified in all instances by the term “approximately” or “about”. At the very least, and not as an attempt to limit the application of the doctrine of equivalents to the claims, each numerical parameter recited in the specification or claims which is modified by the term “approximately” or “about” should at least be construed in light of the number of recited significant digits and by applying rounding techniques. Moreover, all ranges disclosed herein are to be understood to encompass and provide support for claims that recite any and all sub-ranges or any and all individual values subsumed therein. For example, a stated range of 1 to 10 should be considered to include and provide support for claims that recite any and all subranges or individual values that are between and/or inclusive of the minimum value of 1 or more and ending with a maximum value of 10 or less (e.g., 5.5 to 10, 2.34 to 3.56, and so forth) or any value from 1 to 10 (e.g., 3, 5.8, 9.9994, and so forth).

The above detailed descriptions of embodiments of the disclosure are not intended to be exhaustive or to limit the disclosure to the precise form disclosed above. Although specific embodiments of, and examples for, the disclosure are described above for illustrative purposes, various equivalent modifications are possible within the scope of the disclosure, as those skilled in the relevant art will recognize. For example, while steps are presented in a given order, alternative embodiments may perform steps in a different order. While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. The description is thus to be regarded as illustrative instead of limiting.

As discussed herein, percentages, ratios, and/or proportions can be by weight, volume, moles, and/or another measure.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A method, comprising:
extracting a protein solution from a plant substrate;
forming protein micelles, each comprising an agglomerated structure comprising a plurality of protein oligomers, wherein each protein oligomer comprises two or more protein chains; and
producing a plant-based milk using the protein micelles.

2. The method of claim 1, wherein the plant-based milk excludes added polysaccharides.

3. The method of claim 1, further comprising forming a dairy product analog with the plant-based milk, using standard processes from dairy product production.

4. The method of claim 3, wherein the dairy product comprises a cheese.

5. The method of claim 1, further comprising:
inoculating the plant-based milk with a microbial culture;
curdling the inoculated plant-based milk; and
pressing a resultant curd to obtain a plant-based cheese.

6. The method of claim 5, wherein curdling the inoculated plant-based milk comprises:
acidifying the plant-based milk; and
incubating the acidified, inoculated plant-based milk.

7. The method of claim 1, further comprising producing a plant-based cheese using the plant-based milk, wherein producing the cheese comprises heating the plant-based milk to a temperature below 32° C.

8. The method of claim 1, wherein producing the plant-based milk comprises emulsifying the protein micelles, a lipid, and an aqueous solution.

9. The method of claim 1, wherein forming the protein micelles comprises rapidly diluting the protein solution.

10. The method of claim 9, wherein the protein micelles are diluted at room temperature.

11. The method of claim 1, wherein the protein oligomers comprise hexamers, wherein the protein chain comprises a globulin.

12. The method of claim 11, wherein the globulin comprises an 11S globulin.

13. The method of claim 1, wherein the protein micelle consists essentially of proteins and solvent.

14. A food product, comprising:
an emulsion of a lipid and protein micelles, wherein each protein micelle comprises an agglomerated structure comprising a plurality of protein oligomers; and
a cheese culture.

15. The food product of claim 14, wherein the protein oligomers comprise quaternary protein structures, wherein each protein micelle consists essentially of agglomerated quaternary protein structures and a solvent.

16. The food product of claim 14, wherein the protein oligomers comprise hexamers of globulins.

17. The food product of claim 14, wherein the globulins are 11S globulins.

18. The food product of claim 14, wherein the cheese culture comprises at least one of: Penicillium roqueforti. Lactobacillus bulgaricus, Lactobacillus acidophilus, Lactobacillus helveticus, and Lactobacillus lactis, Streptococcus thermophilus, Lactobacillus delbrueckii, Streptococcus lactis, Streptococcus cremoris, Streptococcus lactis subspecies diacetylactis, Leuconostoc citovorum, or Leuconostoc dextranicum.

19. The food product of claim 14, wherein the food product excludes added polysaccharide thickeners and animal proteins.

20. The food product of claim 14, wherein the food product further comprises plant protein solids suspended within the emulsion.

21. The food product of claim 14, wherein the protein oligomers comprise plant proteins, wherein the plant proteins are extracted from at least one of: tree nuts, legumes, squash seeds, or aster seeds.
